(19) United States
(12) Patent Application Publication
(10) Pub. No.: US 2026/0273210 A1
(43) Pub. Date: Sep. 17, 2026

(54) PHYSIOLOGIC CLOSED-LOOP CONTROL FOR A VENTILATOR THAT ADAPTS TO PATIENT RESPONSE TIME

(71) Applicants: ZOLL Medical Corporation, Chelmsford, MA (US); University of Iowa Research Foundation, Iowa City, IA (US)

(72) Inventors: George Beck, Salem, MA (US); Richard D. Branson, Buford, SC (US); Paolo Giacometti, North Grafton, MA (US); Brian P. Harvey, Belmont, MA (US); Jacob Herrmann, Iowa City, IA (US); David W. Kaczka, Iowa City, IA (US); Dorian LeCroy, Parsippany, NJ (US); Joshua W. Lampe, Groton, MA (US); Frency Varghese, Westford, MA (US)

(73) Assignees: ZOLL Medical Corporation, Chelmsford, MA (US); University of Iowa Research Foundation, Iowa City, IA (US)

(21) Appl. No.: 19/081,417

(22) Filed: Mar. 17, 2025

Related U.S. Application Data
(60) Provisional application No. 63/571,051, filed on Mar. 28, 2024.

(51) Int. Cl. A61M 16/00; G16H 20/40; G16H 40/63

(52) U.S. Cl. CPC A61M 16/024; A61M 16/0003; G16H 20/40; G16H 40/63; A61M 2202/0007; A61M 2202/02; A61M 2205/3303; A61M 2205/50; A61M 2230/005; A61M 2230/20

(57) ABSTRACT

A peripheral oxygen saturation (SpO₂) response time (also referred to as a patient response time) may be determined automatically by a ventilator and used as part of a process for controlling the inspired oxygen fraction (FIO₂) level delivered to the patient. Accordingly, the SpO₂ response time for a given patient can be determined and used to adjust how quickly the FIO₂ level is changed (e.g., more quickly for a patient having a shorter response time and more slowly for a patient having a longer response time). The ventilator may enter a particular operation mode (e.g., a response time procedure) to determine the patient response time. The ventilator may be designed to pause closed loop control of the FIO₂ level and execute the response time procedure to determine the patient response time. The ventilator may return to FIO₂ closed loop control at the conclusion of the response time procedure.

### CROSS-REFERENCE TO RELATED APPLICATIONS

This application claims the benefit of U.S. Provisional Application No. 63/571,051, filed Mar. 28, 2024, the disclosure of which is incorporated by reference herein in its entirety.

### BACKGROUND

Providing appropriate mechanical ventilation to individuals as well as optimizing aspects of the provided ventilation, can pose critical challenges, particularly in pre-hospital or other non-hospital settings, among others. In non-hospital settings, for example, care providers may have limited experience or training relating to ventilation, which can challenge their ability to provide proper care given the patient and their disease state consistent with the standard of care. Yet optimizing ventilation, which can include tracking and continuous adjustment of a number of ventilation related parameters, can be crucial to the patient's care and even survival.

Given the foregoing, it is perhaps unsurprising that, unfortunately, suboptimal or injurious ventilation practices and patterns have been common, particularly in non-hospital or pre-hospital settings where caregivers who are inexperienced in advanced life support are tasked with providing ventilation to the patient. These may include, for example, inappropriate management of supplemental oxygen, inadequate or injurious tidal volume, and nontherapeutic administration of positive end-expiratory pressure (PEEP), among other things. As such, for example, providing or facilitating providing safe, optimized mechanical ventilation, particularly (although not only) in non-hospital and pre-hospital settings and with less intensely ventilation-trained care providers, continues to impact the outcome of those patients who require mechanical ventilator support outside of the hospital.

### SUMMARY

According to an embodiment of the present disclosure, a mechanical ventilator apparatus for customizing therapeutic delivery of gas to a patient includes a gas delivery apparatus, having a patient interface, configured to deliver the gas to the patient; an oximetry sensor configured to generate signals representative of an oxygen concentration of the patient's blood; and a controller, comprising a processor and a memory, in communication with the gas delivery apparatus and the oximetry sensor. The controller is configured to receive the signals representative of the oxygen concentration of the patient's blood, determine the oxygen concentration of the patient's blood based at least in part on the received signals, provide a test change in an FIO₂ setting of the gas being delivered to the patient, determine a patient response time based at least in part on the oxygen concentration of the patient's blood in response to the test change in the FIO₂ setting, and adjust the FIO₂ setting based at least in part on the determined patient response time and the oxygen concentration of the patient's blood.

Some implementations may include one or more of the following features. The patient response time may be specific to the patient. The controller may be configured to determine an FIO₂ correction value and adjust the FIO₂ setting based at least in part on the FIO₂ correction value. The FIO₂ correction value may be inversely proportional to the patient response time.

The FIO₂ correction value may include a proportional gain term and a derivative gain term. The controller may be configured to determine the derivative gain term by determining a rate at which an SpO₂ level representative of the oxygen concentration of the patient's blood changes over a set period of time. The controller may be configured to determine the proportional gain term by determining both a response time coefficient representative of the patient response time and an error term representative of a difference between an SpO₂ level representative of the oxygen concentration of the patient's blood and a target SpO₂ level. The controller may be configured to apply a first proportional gain coefficient to the response time coefficient and error term if the SpO₂ level is within a predetermined range, and may be configured to apply a second proportional gain coefficient to the response time coefficient and error term if the SpO₂ level is outside of the predetermined range. The predetermined range may be between 93% and 99%. The second proportional gain coefficient may be twice the magnitude of the first proportional gain coefficient.

The controller may be configured to provide the test change by controlling the gas delivery apparatus to change an FIO₂ setting of the gas being delivered to the patient from a first FIO₂ level to a second FIO₂ level. The controller may be configured to determine the patient response time in response to determining that the first FIO₂ level is less than a max FIO₂ level, and that a SpO₂ level representative of the oxygen concentration of the patient's blood is less than a max SpO₂ level. The controller may be configured to determine the patient response time based at least in part on a period between a first time corresponding to the change in the FIO₂ setting from the first FIO₂ level to the second FIO₂ level and a second time corresponding to a specified response in the oxygen concentration of the patient's blood. The period may be a time period. The first time may correspond to a beginning of a measurement period used to determine the patient response time. The period may be a first period and the controller may be further configured to control the gas delivery apparatus to change the FIO₂ setting from the second FIO₂ level to a third FIO₂ level, determine a second period between a third time corresponding to the change in the FIO₂ setting from the second FIO₂ level to the third FIO₂ level and a fourth time corresponding to a specified response in the oxygen concentration of the patient's blood, and determine the patient response time based at least in part on the first period and the second period. The second FIO₂ level may be greater than the first FIO₂ level, and the third FIO₂ level may be less than the second FIO₂ level. The specified response in the oxygen concentration of the patient's blood may be based on a percentage of a total change in a measured SpO₂ level representative of the oxygen concentration of the patient's blood between the first time and a third time when the measured SpO₂ level reaches a steady state. The SpO₂ level may reach a steady state when the SpO₂ level has not substantially changed over a given period of time. The specified response in the oxygen concentration of the patient's blood may be a peak response in a measured SpO₂ level representative of the oxygen concentration of the patient's blood. The peak response in the measured SpO₂ level may be determined using a regression-based model fit to data representative of the measured SpO₂ level. The specified response in the oxygen concentration of the patient's blood may be a given change in a measured SpO₂ level representative of the oxygen concentration of the patient's blood. The given change in the measured SpO₂ level may be determined using a regression-based model fit to data representative of the measured SpO₂ level.

The controller may be further configured to determine if the oxygen concentration of the patient's blood has been below a threshold value for a predetermined period of time. In response to determining that the oxygen concentration of the patient's blood has been below the threshold value for the predetermined period of time, the controller may be further configured to control the gas delivery apparatus to change the FIO₂ setting to an FIO₂ level between 50% and 100%.

The oximetry sensor may include a pulse oximetry sensor comprising an SpO₂ sensor. The pulse oximetry sensor may be configured for attachment to an ear or a finger of the patient. The controller may be configured to adjust the FIO₂ setting to cause a measured SpO₂ level representative of the oxygen concentration of the patient's blood to approach a target SpO₂ level.

According to an embodiment of the present disclosure, a mechanical ventilator apparatus includes a gas delivery apparatus, having a patient interface, configured to deliver gas to a patient; an oximetry sensor configured to generate signals representative of an oxygen concentration of the patient's blood; and a controller, comprising a processor and a memory, in communication with the gas delivery apparatus and the oximetry sensor. The controller is configured to control the gas delivery apparatus to deliver the gas to the patient according to an FIO₂ setting, receive the signals representative of the oxygen concentration of the patient's blood from the oximetry sensor, change the FIO₂ setting to a first FIO₂ level, determine a first time period over which an SpO₂ level associated with the oxygen concentration of the patient's blood changes in response to changing the FIO₂ setting to the first FIO₂ level, change the FIO₂ setting to a second FIO₂ level, determine a second time period over which the SpO₂ level changes in response to changing the FIO₂ setting to the second FIO₂ level, and determine a patient response time based at least in part on the first time period and the second time period.

Some implementations may include one or more of the following features. The controller may be configured to determine the patient response time as being the greater of the first time period or the second time period, as being the mean between the first time period and the second time period, or as being the lesser of the first time period or the second time period.

The controller may be configured to change the FIO₂ setting to the first FIO₂ level in response to determining that a current FIO₂ level is less than a max FIO₂ level, and that the SpO₂ level is less than a max SpO₂ level. The first FIO₂ level may be greater than the current FIO₂ level, and the second FIO₂ level may be less than the first FIO₂ level.

The controller may be configured to change the FIO₂ setting to the first FIO₂ level in response to determining that a current FIO₂ level is greater than a min FIO₂ level, and that the SpO₂ level is greater than a min SpO₂ level. The first FIO₂ level may be less than the current FIO₂ level, and the second FIO₂ level may be greater than the first FIO₂ level.

The first time period may be based at least in part on a first time that it takes for the SpO₂ level to reach a steady state, and the second time period may be based at least in part on a second time that it takes for the SpO₂ level to reach a steady state. The SpO₂ level may reach a steady state when the SpO₂ level has not substantially changed over a given period of time. The SpO₂ level may reach a steady state when the SpO₂ level has not changed more than 1% over a period of 1 minute. The SpO₂ level may reach a steady state when a derivative of the SpO₂ level is below a threshold value over the given period of time. The first time period may be a first fraction of the first time and the second time period may be a second fraction of the second time.

The controller may be further configured to receive additional signals representative of the oxygen concentration of the patient's blood from the oximetry sensor during delivery of the gas to the patient, determine a current oxygen concentration of the patient's blood based at least in part on the received additional signals, and based at least in part on the current oxygen concentration of the patient's blood and the patient response time, control the gas delivery apparatus to adjust the FIO₂ setting.

According to an embodiment of the present disclosure, a method for controlling mechanical ventilation being provided to a patient using a mechanical ventilator apparatus having a controller configured to perform operations of the method includes receiving signals representative of an oxygen concentration of the patient's blood; determining the oxygen concentration of the patient's blood based at least in part on the received signals; providing a test change in an FIO₂ setting of the mechanical ventilator apparatus to control gas being delivered to the patient; determining a patient response time based at least in part on the oxygen concentration of the patient's blood in response to the test change in the FIO₂ setting; and controlling the mechanical ventilator apparatus to adjust the FIO₂ setting based at least in part on the determined patient response time and the oxygen concentration of the patient's blood.

Some implementations may include one or more of the following features. The method may further include determining an FIO₂ correction value, and adjusting the FIO₂ setting of the mechanical ventilator apparatus based at least in part on the FIO₂ correction value. The FIO₂ correction value may be inversely proportional to the patient response time. Adjusting the FIO₂ setting may cause a measured SpO₂ level representative of the oxygen concentration of the patient's blood to approach closer to a target SpO₂ level.

The FIO₂ correction value may include a proportional gain term and a derivative gain term. The method may further include determining the derivative gain term by determining a rate that an SpO₂ level representative of the oxygen concentration of the patient's blood changes over a set period of time. The method may further include determining the proportional gain term by determining both a response time coefficient representative of the patient response time and an error term representative of a difference between an SpO₂ level representative of the oxygen concentration of the patient's blood and a target SpO₂ level. The method may further include applying a first proportional gain coefficient to the response time coefficient and error term if the SpO₂ level is within a predetermined range, and applying a second proportional gain coefficient to the response time coefficient and error term if the SpO₂ level is outside of the predetermined range. The predetermined range may be between 93% and 99%. The second proportional gain coefficient may be twice the magnitude of the first proportional gain coefficient.

In the method, determining the patient response time may be performed in response to determining that a current FIO₂ level is less than a max FIO₂ level, and that a SpO₂ level representative of the oxygen concentration of the patient's blood is less than a max SpO₂ level.

In the method, providing the test change may include controlling the mechanical ventilator apparatus to change an FIO₂ setting of the gas being delivered to the patient from a first FIO₂ level to a second FIO₂ level. Determining the patient response time may include determining a time period between a first time corresponding to the change in the FIO₂ setting from the first FIO₂ level to the second FIO₂ level and a second time corresponding to a specified response in the oxygen concentration of the patient's blood. The specified response in the oxygen concentration of the patient's blood may be based on a percentage of a total change in the measured oxygen concentration of the patient's blood between the first time and a third time when the measured oxygen concentration of the patient's blood reaches a steady state. The time period may be a first time period and determining the patient response time may further include controlling the mechanical ventilator apparatus to change the FIO₂ setting from the second FIO₂ level to a third FIO₂ level, determining a second time period between a third time corresponding to the change in the FIO₂ setting from the second FIO₂ level to the third FIO₂ level and a fourth time corresponding to a specified response in the oxygen concentration of the patient's blood, and determining the patient response time based at least in part on the first time period and the second time period. The second FIO₂ level may be greater than the first FIO₂ level, and the third FIO₂ level may be less than the second FIO₂ level.

The method may further include determining if the oxygen concentration of the patient's blood has been below a threshold value for a predetermined period of time. In response to determining that the oxygen concentration of the patient's blood has been below the threshold value for the predetermined period of time, the method may further include controlling the mechanical ventilator apparatus to adjust the FIO₂ setting to an FIO₂ level between 50% and 100%.

In the method, controlling the mechanical ventilator apparatus to adjust the FIO₂ setting may cause a measured SpO₂ level representative of the oxygen concentration of the patient's blood to approach a target SpO₂ level. Controlling the mechanical ventilator apparatus to adjust the FIO₂ setting may include adjusting the FIO₂ setting to an updated FIO₂ level and the method may further include controlling the mechanical ventilator apparatus to change a PEEP setting to a current PEEP value, and based at least in part on the updated FIO₂ level, controlling the mechanical ventilator apparatus to adjust the PEEP setting to an updated PEEP value.

According to an embodiment of the present disclosure, a mechanical ventilator apparatus includes a gas delivery apparatus, having a patient interface, configured to deliver gas to a patient, an oximetry sensor configured to generate signals representative of an oxygen concentration of the patient's blood, and a controller, comprising a processor and a memory, in communication with the gas delivery apparatus and the oximetry sensor. The controller is configured to control the gas delivery apparatus to deliver gas to the patient according to an FIO₂ setting, receive the signals representative of the oxygen concentration of the patient's blood from the oximetry sensor during delivery of the gas to the patient, determine the oxygen concentration of the patient's blood based at least in part on the received signals, obtain a patient response time or a parameter associated with the patient response time, and based at least in part on the oxygen concentration of the patient's blood and the patient response time, control the gas delivery apparatus to adjust the FIO₂ setting. The patient response time is based at least in part on a time period between a first time corresponding to a change in the FIO₂ setting and a second time corresponding to a specified response in the oxygen concentration of the patient's blood.

Some implementations may include one or more of the following features. The patient response time may be specific to the patient. The specified response in the oxygen concentration of the patient's blood may be based on a percentage of a total change in the measured oxygen concentration of the patient's blood between the first time and a third time when the measured oxygen concentration of the patient's blood reaches a steady state.

The controller may be configured to determine an FIO₂ correction value and to adjust the FIO₂ setting based at least in part on the FIO₂ correction value. The FIO₂ correction value may include a proportional gain term and a derivative gain term. The controller may be configured to determine the derivative gain term by determining a rate that an SpO₂ level representative of the oxygen concentration of the patient's blood changes over a set period of time. The controller may be configured to determine the proportional gain term by determining both a response time coefficient representative of the patient response time and an error term representative of a difference between an SpO₂ level representative of the oxygen concentration of the patient's blood and a target SpO₂ level. The controller may be configured to apply a first proportional gain coefficient to the response time coefficient and error term if the SpO₂ level is within a predetermined range, and may be configured to apply a second proportional gain coefficient to the response time coefficient and error term if the SpO₂ level is outside of the predetermined range. The predetermined range may be between 93% and 99%. The second proportional gain coefficient may be twice the magnitude of the first proportional gain coefficient.

The controller may be further configured to determine if the oxygen concentration of the patient's blood has been below a threshold value for a predetermined period of time. In response to determining that the oxygen concentration of the patient's blood has been below the threshold value for the predetermined period of time, the controller may be further configured to control the gas delivery apparatus to change the FIO₂ setting to an FIO₂ level between 50% and 100%.

The oximetry sensor may include a pulse oximetry sensor comprising an SpO₂ sensor. The pulse oximetry sensor may be configured for attachment to an ear or a finger of the patient. The controller may be configured to adjust the FIO₂ setting to cause a measured SpO₂ level representative of the oxygen concentration of the patient's blood to approach a target SpO₂ level. The patient response time or the parameter associated with the patient response time may be obtained via the patient interface of the gas delivery apparatus.

According to an embodiment of the present disclosure, a mechanical ventilator apparatus includes a gas delivery apparatus, having a patient interface, configured to deliver gas having a fraction of inspired oxygen to a patient, an oximetry sensor configured to generate signals representative of an oxygen concentration of the patient's blood, and a controller, comprising a processor and a memory, in communication with the gas delivery apparatus and the oximetry sensor. The controller is configured to change the fraction of inspired oxygen of the gas to a new fraction of inspired oxygen, determine an amount of time required for the oxygen concentration of the patient's blood to achieve a specified response to the new fraction of inspired oxygen, and control subsequent delivery of the gas based at least in part on the amount of time required for the oxygen concentration of the patient's blood to achieve the specified response.

Some implementations may include one or more of the following features. The controller may be configured to control subsequent delivery of the gas by adjusting an FIO₂ setting of the gas delivery apparatus. The controller may be configured to determine an FIO₂ correction value and to adjust the FIO₂ setting based at least in part on the FIO₂ correction value. The controller may be configured to adjust the FIO₂ setting to cause a measured SpO₂ level representative of the oxygen concentration of the patient's blood to approach a target SpO₂ level. The oximetry sensor may include a pulse oximetry sensor comprising an SpO₂ sensor.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, identical or nearly identical components that are illustrated in various figures may be represented by like numerals. For purposes of clarity, not every component may be labeled in every figure. Herein, where a single element is recited, it is implied that one or more may be included in various embodiments.

FIG. 1 illustrates an example system incorporating physiologic closed-loop control (PCLC) with a response time procedure in mechanical ventilation of a patient, in accordance with some embodiments of the present disclosure.

FIG. 2 is a graphical illustration showing how the controller changes FIO₂ for three different response time coefficients and the resultant changes in SpO₂ of a simulated patient having different response times in accordance with some embodiments of the present disclosure.

FIG. 3 is a block diagram of different operation periods and procedures of therapy of ventilator operation, including a response time procedure, in accordance with some embodiments of the present disclosure.

FIGS. 4A-4C are flow diagrams that illustrate example physiologic closed-loop operation phases for a ventilator, in accordance with some embodiments of the present disclosure.

FIGS. 5A and 5B are graphical illustrations for determining the value of a response time coefficient used in the ventilator physiologic closed-loop control system that includes automated control of FIO₂, in accordance with some embodiments of the present disclosure.

FIGS. 6A and 6B are graphical illustrations for determining the value of a proportional gain coefficient used in the ventilator physiologic closed-loop control system that includes automated control of FIO₂, in accordance with some embodiments of the present disclosure.

FIGS. 7A and 7B are graphical illustrations for determining the value of a derivative gain coefficient used in the ventilator physiologic closed-loop control system that includes automated control of FIO₂, in accordance with some embodiments of the present disclosure.

FIGS. 8A-8C are graphical illustrations for determining particular coefficients based on measured parameters for use in the ventilator physiologic closed-loop control system that includes automated control of FIO₂, in accordance with some embodiments of the present disclosure.

FIG. 9 is a block diagram of a patient response time procedure used by a ventilator, in accordance with some embodiments of the present disclosure.

FIG. 10 is a flow diagram of a patient response time procedure used by a ventilator, in accordance with some embodiments of the present disclosure.

FIG. 11A is a graphical illustration of tracking changes in an SpO₂ level for a step increase in an FIO₂ level for different patient response times, in accordance with some embodiments of the present disclosure.

FIG. 11B is a magnified portion of the graphical illustration from FIG. 11A.

FIG. 11C is another magnified portion of the graphical illustration from FIG. 11A.

FIG. 11D is a graphical illustration of tracking changes in an SpO₂ level for a step decrease in an FIO₂ level for different patient response times, in accordance with some embodiments of the present disclosure.

FIG. 11E is a magnified portion of the graphical illustration from FIG. 11D.

FIG. 12A is another graphical illustration of tracking changes in an SpO₂ level for a shorter step change in an FIO₂ level for different patient response times, in accordance with some embodiments of the present disclosure.

FIG. 12B is a graphical illustration of using a model to fit the measured SpO₂ data when changing the FIO₂ level, in accordance with some embodiments of the present disclosure.

FIG. 13 illustrates an example portable ventilation system incorporating physiologic closed-loop control system that includes automated control of FIO₂ having a patient response time procedure, in accordance with some embodiments of the present disclosure.

FIG. 14 is a flow diagram illustrating an example method of operation of an initiation period of a ventilation system incorporating physiologic closed-loop control system that includes automated control of FIO₂, in accordance with some embodiments of the present disclosure.

FIG. 15 is a flow diagram illustrating an example method of operation of an example test breaths period of a ventilation system incorporating physiologic closed-loop control system that includes automated control of FIO₂, in accordance with some embodiments of the present disclosure.

FIG. 16 is a flow diagram illustrating aspects of an example method of operation of a desaturation related process, in accordance with some embodiments of the present disclosure.

FIG. 17 illustrates a simplified example portable ventilator and display, with FIO₂ physiologic closed-loop control enabled, in accordance with some embodiments of the present disclosure.

FIG. 18 illustrates aspects of an example pneumatic system that can be used with a portable ventilator, in accordance with some embodiments of the present disclosure.

FIG. 19 illustrates aspects of example external gas supply systems that can be used with a portable ventilator, in accordance with some embodiments of the present disclosure.

FIG. 20 illustrates aspects of example patient circuits that can be used with a portable ventilator, in accordance with some embodiments of the present disclosure.

### DETAILED DESCRIPTION

Overall, optimization in view of pre-hospital care related factors can favor increased simplicity, increased stability and a generally more limited or conservative approach with regard to ventilation parameter value determinations and adjustments. In some cases, frequent user checks and confirmations, user warnings, or user alerts are used to ensure the ventilator system is operating correctly or within certain parameters. Some approaches to providing closed loop control favor greater overall stability, less frequent changes in parameters, and more or greater change in conditions required for changes in parameter values. Accordingly, such approaches include use of more conservative parameter values, or more conservative high or low limits, such as with regard to parameters that may present a potentially high degree of patient risk.

In various embodiments, the fraction of inspired oxygen (FIO₂) provided to a patient by a ventilator system may be continuously adjusted based on a measured patient peripheral oxygen saturation (SpO₂), which is used as an indicator of patient blood oxygen saturation. Generally, a lower SpO₂, or decreasing SpO₂ (as may be determined, for example, based on a single or current SpO₂ measurement, or several SpO₂ measurements over a recent period of time) may tend to indicate a patient with more impaired lung or respiratory system dysfunction, who may be in need of more oxygenation support. In some embodiments, generally, when a patient's SpO₂ is below a target level (such as a pre-determined SpO₂ value), and potentially also based at least in part on how much below, and/or a rate of decrease, FIO₂ may procedurally tend to be increased in order to increase support of a patient's oxygenation. When SpO₂ is above the target level, and potentially also based at least in part on how much above, FIO₂ may procedurally tend to be decreased, since the patient may not need of as much oxygenation support. As such, FIO₂ may be increased or decreased based at least in part on the need of the patient as indicated at least in part by SpO₂ and/or, for example, some other indication(s), such as one or more other non-invasively sensed, measured or determined indications of patient oxygen saturation. Procedurally determined factors, such as derivative gain and proportional gain factors, may affect a direction (increase or decrease) and amount of FIO₂ adjustment. Specifically with regards to the closed loop control of the FIO₂ provided to a patient by a ventilator system, changes to the FIO₂ levels may be performed at a relatively slow rate to bring the measured SpO₂ from its current level to a target level. The slow rate of change to the ventilator's FIO₂ level may be done in order to safely accommodate all patients, including those with slower response times to the change in the FIO₂ level.

However, this conservative approach to adjusting the FIO₂ level can have drawbacks. Primarily, the FIO₂ level may be changed at a slower rate than necessary for certain patients. This occurs because not all patients have the same SpO₂ response time (also referred to as a patient response time). The SpO₂ response time is the delay between a change in oxygenation in the lungs (generally considered to be the same time that a change is made to the FIO₂ level or the time when a next breath is taken after changing the FIO₂ level) and when that change is reflected in a measurement of blood oxygen saturation at the measurement site (e.g., a pulse oximeter measurement made at a finger or earlobe). A patient's SpO₂ response time is dependent on several factors, including the measurement site used, patient cardiopulmonary physiology, and vasoconstriction or vasodilation. Accordingly, the SpO₂ response time is specific to the patient. A ventilator using physiologic closed-loop control for its FIO₂ output may be designed to assume that all patients have a relatively long patient response time to conservatively ensure that the changing FIO₂ level does not move too quickly for the measurement to keep up, which could cause undesirable oscillations in the output. But this approach also causes patients that do have faster SpO₂ response times to endure unnecessarily slow adjustments to their SpO₂ level. As an example, for a patient with a current SpO₂ of 96% and a target SpO₂ of 94%, certain ventilator physiologic closed-loop control processes that do not consider the SpO₂ response time may take approximately 19 minutes to wean the FIO₂ setting on the ventilator from 100% to 21% to respectively bring the patient's SPO₂ from 96% to 94%. In the pre-hospital settings described above, this delay in achieving the appropriate level of oxygenation support not only affects patient care but can also waste the finite oxygen supply available to support the patient.

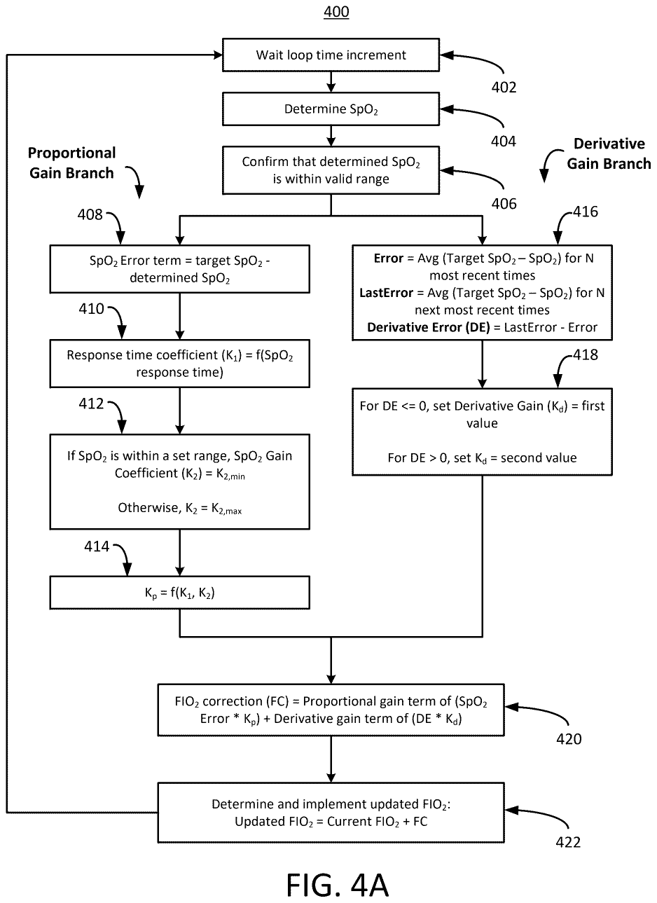

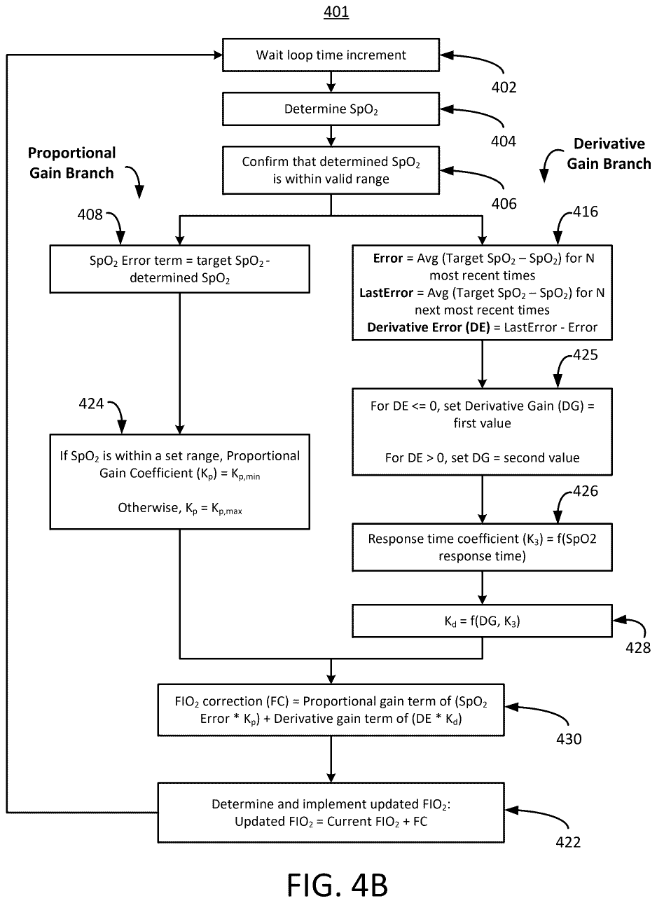

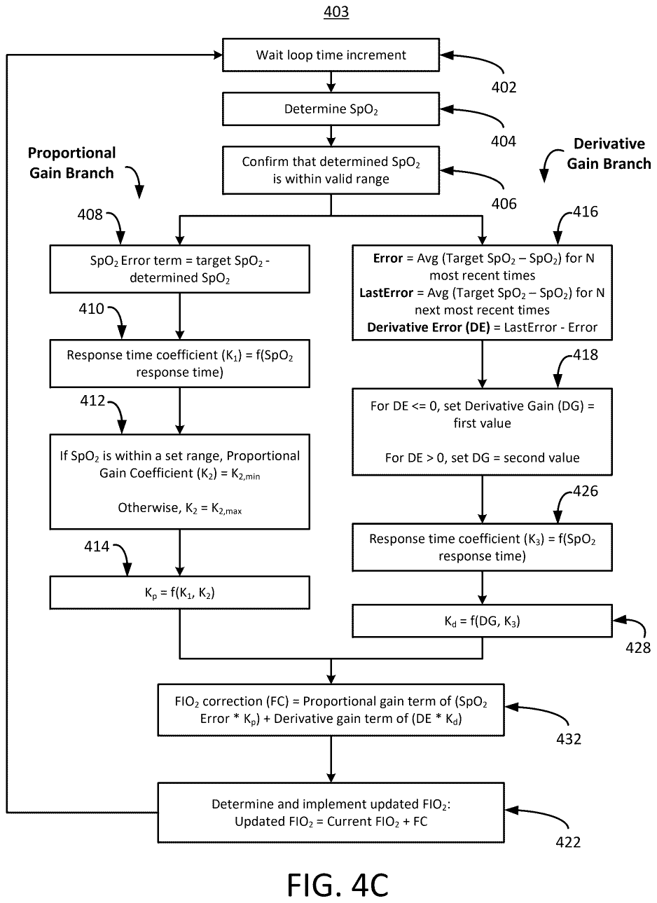

According to some embodiments, the SpO₂ response time may be determined automatically by the ventilator and used in its process for controlling the FIO₂ level. Thus, the SpO₂ response time for a given patient can be determined and used to adjust how quickly the FIO₂ level is changed for that patient (e.g., more quickly for a patient having a fast response time and more slowly for a patient having a slower response time). In some examples, the ventilator uses a particular operational module (e.g., a response time procedure) to determine the patient response time. In some examples, the ventilator may be designed to execute the response time procedure to determine the patient response time prior to any active physiologic closed-loop control of the FIO₂ level. In some examples, the ventilator may be designed to pause physiologic closed-loop control of the FIO₂ level and execute the response time procedure to determine the patient response time. The ventilator may return to physiologic closed-loop control of the FIO₂ level at the conclusion of the response time procedure function. In some examples, the ventilator may be designed to execute the response time procedure to determine the patient response time in response to a manual request received via a user interface on the ventilator or on any computing device communicatively coupled with the ventilator. Regardless of when the response time procedure is executed, the determined patient response time may then be used as a parameter within the physiologic closed-loop control process to adjust the FIO₂ level accordingly. The FIO₂ level may continue to be adjusted at a particular rate based at least in part on the patient response time. Embodiments of processes for physiologic closed-loop control of FIO₂, including use of derivative gain and proportional gain that each consider or that both consider the patient response time, are described with reference to FIGS. 4A-4C.

According to some embodiments, executing the response time procedure involves the ventilator automatically increasing or decreasing the FIO₂ level by a given amount and subsequently monitoring the patient's SpO₂ level in response to the change in FIO₂. There are several ways that the patient's SpO₂ could be monitored to provide an estimation of the patient's response time.

In one example, the amount of time it takes for the patient's SpO₂ level to reach a steady-state condition following the change in FIO₂ can be used to help determine the response time for the patient. In this context, the SpO₂ level is considered to reach a steady state when the SpO₂ level has not changed more than a given percentage over a given period of time. For example, the SpO₂ level may be considered to have reached a steady state if the level has not changed more than 1% over 1 minute, more than 2% over 1 minute, more than 5% over 1 minute, more than 1% over 3 minutes, more than 2% over 3 minutes, more than 5% over 3 minute, more than 1% over 5 minutes, more than 2% over 5 minutes, or more than 5% over 5 minute. Other statistical methods could also be used to determine whether the SpO₂ level has reached a steady state. In some examples, FIO₂ is increased from a first value to a second value and the patient's SpO₂ level is monitored. Once the patient's SpO₂ level reaches a steady state, FIO₂ is decreased from the second value back to the first value and the patient's SpO₂ level is once again monitored. The time that it takes for the patient's SpO₂ level to reach a steady state following the increase of FIO₂ and the time that it takes for the patient's SpO₂ level to reach a steady state following the decrease of FIO₂ can both be factored into the determination of the patient's response time. It should be noted that the FIO₂ setting can also be decreased from the first value to a third value followed by an increase from the third value to the first value. The patient's SpO₂ can be similarly monitored during each of the initial decrease and subsequent increase in FIO₂ to aid in determining the patient response time.

In another example, FIO₂ may be increased and then decreased in a short ‘pulse’ (e.g., for less than 30 seconds, less than 20 seconds, less than 10 seconds, less than 10 breaths, less than 6 breaths, or less than 4 breaths), and the patient's SpO₂ is monitored during the pulse period and for some time afterwards. In some embodiments, the time that it takes for the patient's SpO₂ to reach a peak in response to the FIO₂ pulse may be used as an indication of the patient's response time. In some embodiments, the time that it takes for the patient's SpO₂ to begin to rise above some threshold in response to the FIO₂ pulse may be used as an indication of the patient's response time. To account for signal noise, a regression model or any other suitable data fitting model may be used to smooth out the patient's SpO₂ measurement data and determine with greater accuracy an SpO₂ signal peak or when the SpO₂ rises above a threshold. It should be noted that the FIO₂ can also be decreased and then increased to form a downward ‘pulse’. The patient's SpO₂ can be similarly monitored during the pulse period and for some time afterwards to determine a time to a peak in the decreasing SpO₂ or a time for the SpO₂ to fall below a threshold.

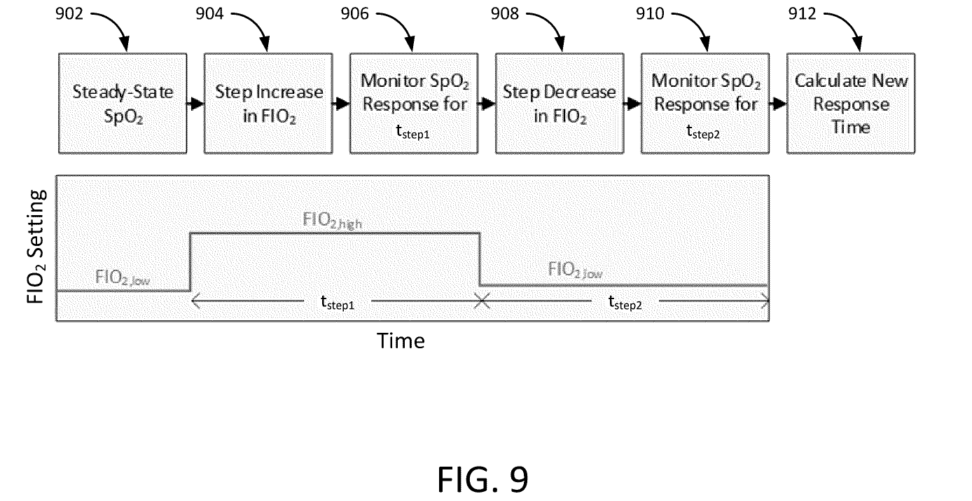

According to some embodiments, the patient response time may be based at least in part on the amount of time it takes for the SpO₂ level to reach a given percentage of the total change in the SpO₂ level caused by the change in the FIO₂ level. As an example, in response to the FIO₂ level increasing from a first level to a second level, the SpO₂ level increases from 85% to 90%. The patient response time may be based on the time it takes for the SpO₂ level to reach 80% of its total change, which in this example would be the time it takes for the SpO₂ level to rise from 85% to 89%. Any other percentages of the total SpO₂ change could be used as well in the determination of the patient response time, such as 75% of the total change (e.g., time it takes for the SpO₂ level to rise from 85% to 88.75%), or 100% of the total change, or even a small change such as anywhere from 5%-25% of the total change. Further details regarding the automatic determination of the patient response time during the response time procedure of the ventilator are described with reference to FIGS. 9-12.

In some embodiments, one or more previously measured SpO₂ values may be taken into account procedurally in determining an FIO₂ adjustment. In particular, in some embodiments, proportional gain and derivative gain may take into account one or more previously measured SpO₂ values. Generally, in some embodiments, derivative gain may take into account an effective rate of decrease in SpO₂ for some period of time up to and including the time of the currently measured SpO₂ (such as may include use of a moving average). Derivative gain may tend to increase the FIO₂ level when SpO₂ is decreasing, such as in a manner that may be associated with, or proportional to, a determined rate of SpO₂ decrease. However, in some embodiments, derivative gain may not be used to decrease FIO₂ when SpO₂ is increasing, and may in this regard be asymmetric in operation. Furthermore, in some embodiments, proportional gain may take into account a determined magnitude of the difference between the current (or current and recent) SpO₂ and a target SpO₂, where a larger difference may lead to a greater adjustment.

In some embodiments, positive end-expiratory pressure (PEEP) is adjusted based at least in part on FIO₂, but also based at least in part on the current PEEP. Since FIO₂ may be adjusted based at least part on patient SpO₂, and since SpO₂ may be associated with how “sick,” compromised or functionally impaired the patient's lungs or respiratory system are, conceptually, FIO₂ may to some degree represent or effectively operate as a surrogate or indicator of how “sick” the patient's lungs or respiratory system are. For example, in some instances, a relatively low and/or decreasing SpO₂ may indicate substantial and/or increasing degree of functional lung impairment, and may result in a relatively high FIO₂. This high FIO₂ may, under appropriate circumstances, warrant and lead to an increase in PEEP (as may be determined with reference to, for example, PEEP change eligibility rules and PEEP selection rules), where PEEP may help essentially open alveoli and support respiratory function. Conversely, in some instances, a relatively high and/or increasing SpO₂ may indicate less substantial and/or decreasing lung impairment (i.e., generally, the patient may be improving, thereby requiring less or no supplement oxygen), and may lead to a relatively low FIO₂, which may, in some instances, warrant and lead to a decrease in PEEP (as may be determined with reference to, for example, PEEP change eligibility rules and PEEP selection rules).

As such, adjusting PEEP based at least in part on FIO₂ may result in PEEP being adjusted based at least in part on an indication of how “sick” or functionally impaired the patient's lungs or respiratory system may be. In some embodiments, various parameters in addition to FIO₂ and PEEP may be continuously adjusted or adjusted using physiologic closed-loop control. These may include, for example, adjustments that may be based at least in part on end-tidal carbon dioxide (EtCO2), which may indicate or suggest normocapnia, hypercapnia or hypocapnia. For example, in some embodiments, parameters including tidal volume (Vt), minute volume (V_{M}) and peak inspiratory pressure (PIP) may be continuously adjusted.

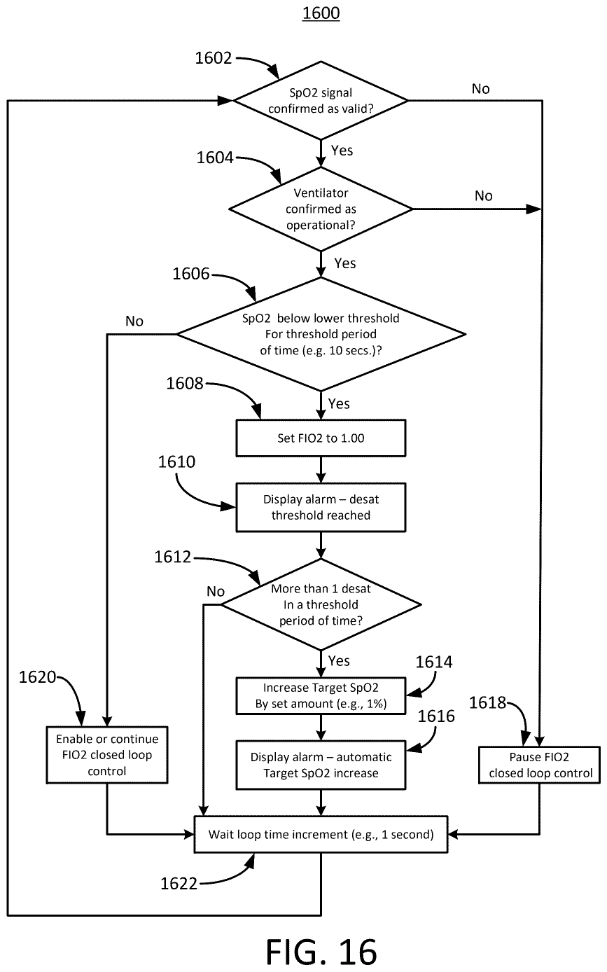

The monitoring of the oxygen concentration of the patient's blood and checking/updating of the FIO₂ setting may be performed on a very frequent basis, such as, e.g., every fraction of a second, every second, every more than one second or several seconds, every minute or several minutes, or based on irregularly or varying duration periods. Monitoring and adjusting of parameters may be implemented or facilitated by one or more controllers that may execute one or more appropriate processes stored in one or more memories. Additionally, optimized initial settings may be determined and utilized. However, in some circumstances, pre-hospital considerations may warrant rapid or increased changes in favor of optimization, such as, for example, increasing FIO₂ to 100% upon detection of a defined patient desaturation condition, as described with reference to FIG. 16.

Herein, in some instances, variations of a term may be utilized that may refer to the same or similar concepts, and certain terms may have meanings that are informed by a particular context. Various ventilation-related terms or abbreviations, including FIO₂, SpO₂, PEEP, and others, may refer to ventilation related settings, even though the word “setting” may or may not be stated, or blood oxygen measurements. Furthermore, reference to a ventilation parameter or parameter setting may be used to refer to the parameter in a conceptual or definitional sense, or the value associated with a particular setting (e.g., “FIO₂ of 95%”). A user, as described herein, may include an individual operating, supervising or in whole or in part responsible for operation of a device such as a portable ventilator in support of the patient's need for respiratory support, even if, during a particular period of time while the device is operating, the user may not be interacting with the device.

An alert or alarm, as used herein, may be presented for the attention of a user, such as by being visually or audibly presented, such as via a display, graphical user interface (GUI) or speaker of a device. However, an alert or alarm may also include reference to alert or alarm conditions that are procedurally identified, recognized or determined by a computerized device and not necessarily presented or displayed. Herein, the term optimizing may include, for example, improvement or improved operation in one or more aspects, for example, relative to an actual, potential or hypothetical less optimized situation or less optimized operation.

Herein, a ventilator can include, for example, a ventilator or ventilation device, apparatus or system, whether or not portable, and whether or not functionality other than ventilation aspects is provided by the device, apparatus or system. Herein, the term adjusting can include changing as well as not changing or maintaining without change, as may be appropriate. Herein, a determined parameter value can include a determined estimated or determined approximated value for the parameter. Herein, the term monitoring can refer to or include, for example, monitoring or tracking performed by a computerized device utilizing one or more processes and not by a person or user, or monitoring by a person or user, or both. Herein, the term continuous can include, among other things, on a periodic basis (with identical or different periods), on a frequent basis, on a repeated basis, or cyclically, for example.

Herein, terms such as hypercapnia, hypocapnia and normocapnia are not intended to be limited to particular ranges or clinical meanings, but are used in a relative sense, such as to indicate relatively high, relatively low, or intermediate, in a particular embodiment, for example. Furthermore, in some embodiments, associated ranges, or some of them, may overlap. In some embodiments, normocapnia may have a lower EtCO₂ threshold of, e.g., in mm Hg, 20, 25, 30, 35, 40, 45 or 50 and an upper threshold of, e.g., in mm Hg, 30, 35, 40, 45, 50, 55 or 60. Hypercapnia may include an EtCO₂ of at or above a threshold of, e.g., in mm Hg, 30, 35, 40, 45, 50, 55 or 60. Hypocapnia may include an EtCO₂ of at or below a threshold of, e.g., in mm Hg, 20, 25, 30, 35, 40, 45 or 50.

The term physiologic closed-loop control, as used herein, may refer to control of one or more ventilation related or patient related parameters, such as with relatively little or no required user action, participation or intervention, and can include reference to, but is not limited to reference to, fully automated or fully automatically regulated control. Physiologic closed-loop control may include, for example, device facilitated or procedurally facilitated tracking, control and adjustment of one or more parameters, which may or may not include user involvement or participation. Where user involvement or participation is included, it may include, for example, confirming a suggested or recommended ventilation setting change or configuration, deciding on implementing a course of action, selecting one of several suggested courses of action, responding to a presented alert or alarm, or other decisions, choices or actions. User involvement or participation could also include, for example, setting or changing a parameter, where a physiologic closed-loop control process proceeds from there, initially according to the user-set or user-changed parameter setting. In various embodiments, if there is user involvement, it may be, for example, among other things, in whole or in part user-initiated, or in whole or in part prompted, suggested, recommended or required.

In some embodiments, physiologic closed-loop control may be utilized but may be subject to manual adjustment or override by the user. For example, in some embodiments, although FIO₂ may be procedurally and automatically controlled by the ventilator based on measured patient SpO₂ and a patient response time, a user may be able to intervene and manually change the FIO₂ setting and/or the patient response time. In some embodiments, following any manual adjustments, physiologic closed-loop control of FIO₂ may resume from that point, at least until any further manual adjustments are made.

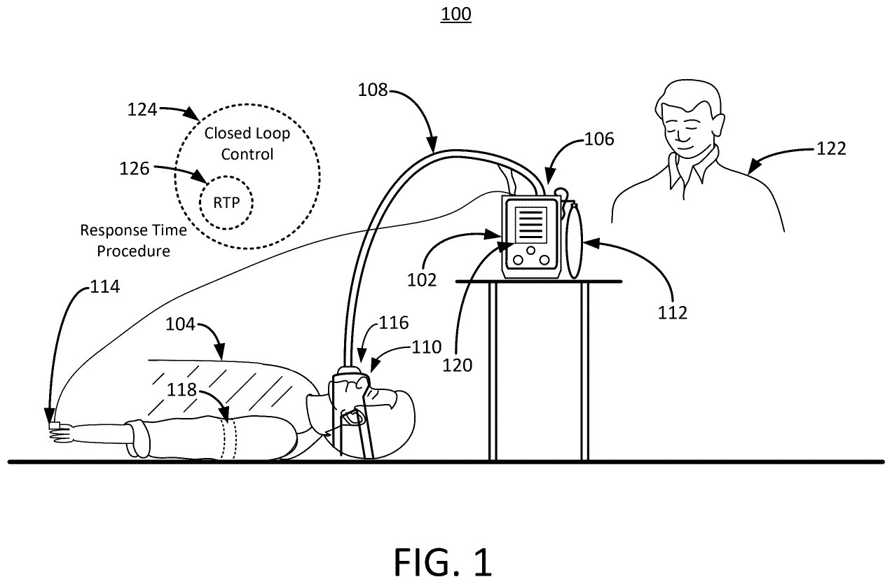

FIG. 1 illustrates an example system 100 incorporating physiologic closed-loop control 124, as conceptually represented by a dashed circle, in mechanical ventilation of a patient 104, in accordance with some embodiments of the present disclosure. While a portable (e.g., may be carried or practically carried or taken by a care provider to a patient at an out-of-hospital or pre-hospital location) ventilator 102 is shown, embodiments are contemplated in which a non-portable ventilator is provided or utilized. When operating, the portable ventilator 102 may provide breathing gas to a patient 104 via a gas delivery apparatus 106, including a patient circuit 108 that includes a facemask 110, though, in some embodiments, ventilation may be provided via intubation rather than via a facemask. A user 122 is also shown. The portable ventilator 102 may be coupled with an oxygen source, such as an oxygen tank 112, and may also be coupled or integrated with other devices. The portable ventilator 102 may also include various sensing, measuring, computerized, electrical, mechanical, coupling and output components. As depicted, the portable ventilator 102 includes an oximetry sensor 114, such as a pulse oximeter or other sensor for providing a direct or indirect measurement, estimation or indication of oxygen saturation (SpO₂) or other blood oxygen content or concentration related parameter, a capnographic sensor 116 or capnograph, and a blood pressure sensor/monitor 118, and may also include one or more flow sensors such as pneumotachometers, or pressure sensors, among other things.

The portable ventilator 102 includes a display and user interface 120 that may provide data relating to various patient physiological, respiratory and ventilation related parameters, and may include other output or presentation components, such as a speaker. In some embodiments, the display and user interface 120, or other output devices, may provide a display that is integrated to include data relating to operation of other coupled devices that may also be in use with the patient, such as, for example, a critical care monitor or monitor/defibrillator. The display and user interface 120 may also allow user interaction, including to obtain or display data, change settings, accept suggested or recommended settings changes, or view or respond to alarms or alerts, among other things.

The portable ventilator 102 is capable of providing physiologic closed-loop control 124 of one or more ventilation or patient related parameters, as described with regard to various embodiments herein. Accordingly, ventilator 102 may include a controller that executes one or more processes in implementing methods described herein. The location or setting depicted in FIG. 1 may include, among other things, a pre-hospital or non-hospital location or setting, which could include an emergency vehicle setting or other venue or location. The portable ventilator 102 may, for example, be stored at or near the location or setting, or may be carried or taken to the location or setting to provide emergency care to the patient 104.

According to some embodiments, the portable ventilator 102 is also capable of executing a response time procedure (RTP) 126 to determine an SpO₂ response time for the patient 104. RTP 126 may be a procedure that is executed within the physiologic closed-loop control 124 process. In some examples, RTP 126 is executed independently of the physiologic closed-loop control 124 process.

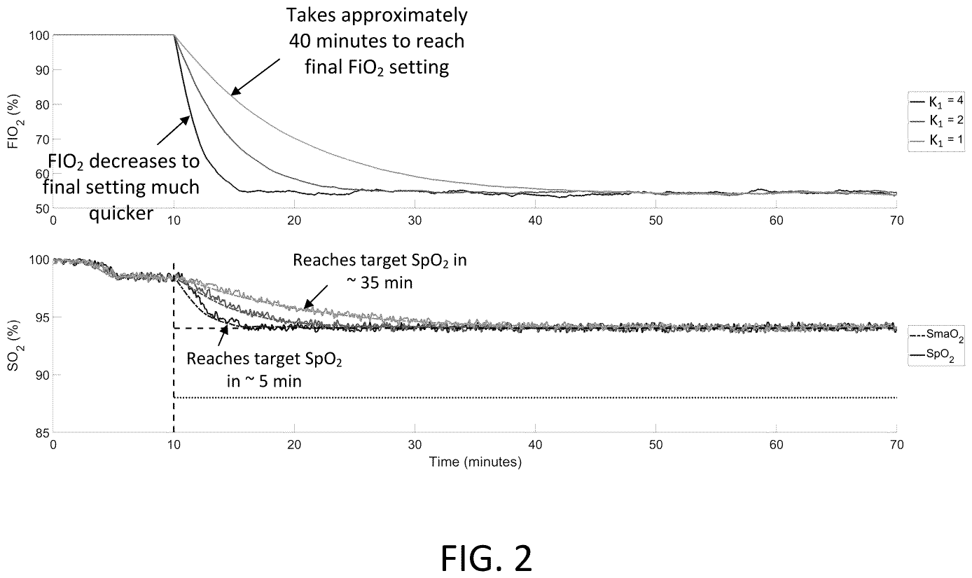

FIG. 2 provides simulated patient SpO₂ levels responding to changes in FIO₂ that highlight the effect the response time coefficient (K₁) has on the outcome. Since the SpO₂ response time (generally reflected by the value of K₁) provides an indication of how quickly the measured SpO₂ level in a patient's blood responds to changes in FIO₂, patients with a faster response time can “keep up” with the higher rate of change of the FIO₂ level. By incorporating the patient response time into the physiologic closed-loop control of the FIO₂ level, those patients with inherently fast response times benefit greatly by reaching their target SpO₂ levels faster compared to patients with slower response times as generally illustrated in FIG. 2.

In this example, the FIO₂ setting on the ventilator starts at 100% and physiologic closed-loop control of FIO₂ is activated at the 10 minute mark. The controller's and patient's behavior for three values of patient response time coefficients (K₁=1, 2, and 4) are compared in the figure. The physiologic closed-loop controller decreases FIO₂ the slowest with a response time coefficient of 1, taking approximately 40 minutes to reach a steady state FIO₂ value of approximately 55%. When the K₁ is equal to 2, the controller decreases FIO₂ more quickly, reaching the steady state FIO₂ value in approximately 15 minutes. When K₁=4, the controller decreases FIO₂ the most quickly, reaching the steady state FIO₂ within approximately 5 minutes. The patient's simulated SpO₂ level also gradually decreases from around 98% at the ten minute mark to target SpO₂ value of 94% in response to the decreasing FIO₂ level. As can be observed, a simulated patient with the highest patient response time coefficient (represented here as a K₁ value of 4) reaches the final SpO₂ level much quicker as compared to having the lowest patient response time coefficient (represented here as a K₁ value of 1). In this example, having the highest response time coefficient causes the final SpO₂ level to be reached in about 5 minutes while having the lowest response time coefficient causes the final SpO₂ level to be reached in about 35 minutes.

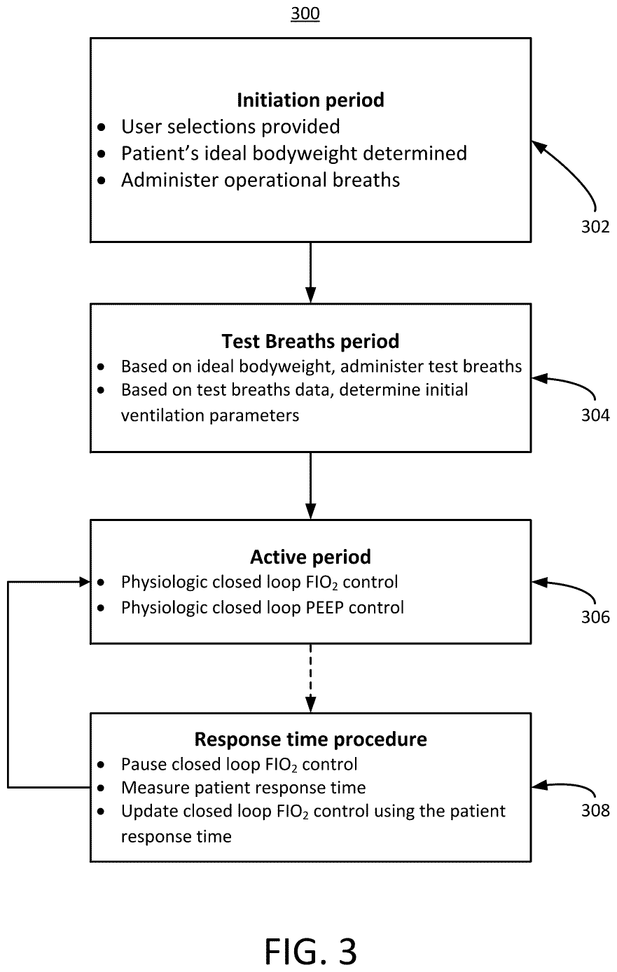

FIG. 3 is a flow diagram illustrating an example method 300 of operation of a ventilation system incorporating physiologic closed-loop control, in accordance with some embodiments of the present disclosure. The method 300 includes an initiation period 302, followed by a test breaths period 304, followed by an active period 306. Method 300 also includes a response time procedure 308 during which a response time procedure can be initiated at any time during the active period 306, after which method 300 returns to the active period 306.

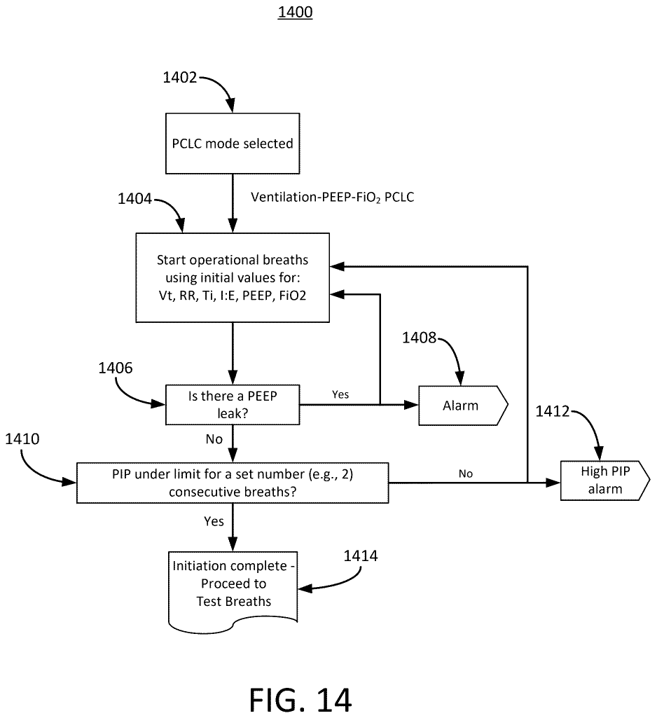

During the initiation period 302, one or more user selections may be made, such as selection of physiologic closed loop control (PCLC) ventilation, although in some embodiments, no such user selection is provided and the type of ventilation is set according to a pre-configured default. A user of the ventilator may be prompted to provide the patient's sex and height, and the patient's “predicted” bodyweight (PBW) may be determined. Based on the patient's PBW, one or more ventilation parameters such as the tidal volume setting to be delivered per breath may be determined. Furthermore, one or several operational breaths are administered to the patient such as, for example, via a gas delivery apparatus 106 and using a facemask 110, as depicted in FIG. 1, coupled with the patient, or using an invasive airway such as an endotracheal tube. Further details associated with initiation period 302 are provided with reference to FIG. 14.

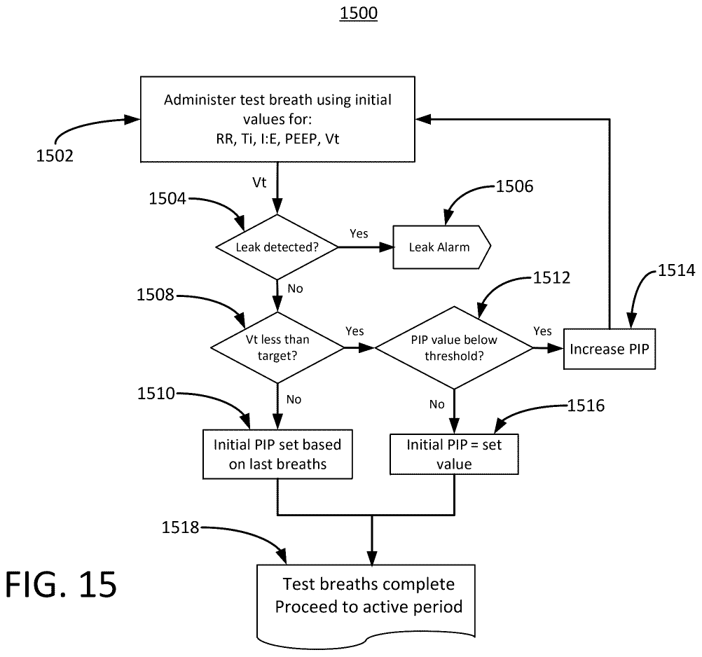

During the test breaths period 304, one or several breaths may be delivered to the patient such as, for example, via a gas delivery apparatus 106 and using a facemask 110, as depicted in FIG. 1, coupled with the patient, or using an invasive airway such as an endotracheal tube, using certain ventilation parameters that are determined based at least in part on the patient's sex and PBW. Based on data associated with the breaths, a set of initial active period ventilation parameters are determined, including, for example, an initial peak inspiratory pressure (PIP) setting. Further details relating to test breaths period 304 are provided with reference to FIG. 15.

During the active period 306, ventilation is provided to the patient such as, for example, via a gas delivery apparatus 106 and using a facemask 110, as depicted in FIG. 1, coupled with the patient, or using an invasive airway such as an endotracheal tube, which may include physiologic closed-loop control of FIO₂, as detailed in embodiments described herein.

At any time during active period 306, method 300 proceeds to response time procedure 308 where the physiologic closed-loop control of the FIO₂ is paused in order to determine a patient response time via execution of the response time procedure. As discussed in more detail herein, the FIO₂ level is increased or decreased by a certain amount and the SpO₂ level of the patient is monitored to observe the patient's response to the change in FIO₂. In general, any change to the FIO₂ level during the response time procedure is considered a test change to the FIO₂ setting of the ventilator. From at least this information, a patient response time can be determined. A response time value may be generated that is associated with the determined patient response time, and the response time value is applied to the physiologic closed-loop control process to affect the rate at which FIO₂ is changed for the given patient based at least in part on the patient's response time.

The transition between active period 306 and response time procedure 308 may occur at predetermined intervals, such as seconds, minutes (e.g., 1-5 minutes, 5-10 minutes, 10-30 minutes, 30-60 minutes) or one to several hours, to ensure that the response time measurement is up to date. In some examples, response time procedure 308 is executed once at or near the start of the physiologic closed-loop control of active period 306 and is not executed again as long as the ventilator remains active on a given patient. This may be sufficient as the patient's response time is unlikely to change significantly over time or if the duration of treatment and/or transport is relatively short. In some examples, active period 306 transitions to response time procedure 308 in response to a manual input received from a user via, for example, user interface 120 as illustrated in FIG. 1.

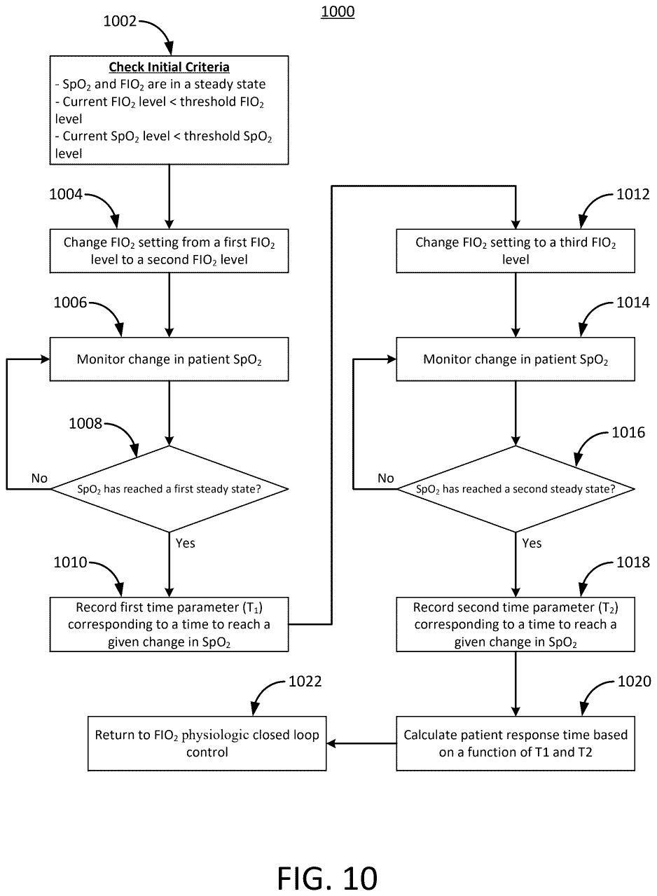

FIGS. 4A-4C are flow diagrams illustrating example methods 400, 401, and 403 of operation of a gain related process, such as an process used by a ventilator during active period 306, in accordance with some embodiments of the present disclosure. Each of FIGS. 4A-4C provides an example of how a proportional gain term and a derivative gain term are used in determining an overall correction that is applied in adjusting FIO₂ based on measured patient SpO₂. In the example method 400 of FIG. 4A, a response time coefficient associated with the patient response time is applied to the proportional gain term to potentially increase the value of the FIO₂ adjustment. In the example method 401 of FIG. 4B, the response time coefficient associated with the patient response time is applied to the derivative gain term to potentially increase the value of the FIO₂ adjustment. In the example method 403 of FIG. 4C, the response time coefficient associated with the patient response time is applied to both the proportional gain term and the derivative gain term to potentially increase the value of the FIO₂ adjustment. The patient response time may be determined by the ventilator during a response time procedure as discussed in more detail with reference to FIGS. 9 and 10. In some embodiments, the patient response time may be obtained manually by the user, such as entered by the user via a user interface on the ventilator, or the patient response time may be obtained from memory in situations where the patient response time has been previously determined and stored in the memory. Regardless of how the patient response time is received, an overview of the physiologic closed-loop control process is provided, followed by a detailed example.

In some embodiments, at a given time, the overall adjustment to the FIO₂ may be based on a correction that is calculated as the sum of a proportional gain term and a derivative gain term. A positive overall correction value may indicate an increase in FIO₂, while a negative overall correction value may indicate a decrease in FIO₂. The proportional gain term may operate to affect the FIO₂ adjustment to create a tendency to draw the patient's (current) SpO₂ toward a Target SpO₂. Therefore, if SpO₂ is below the Target SpO₂, it will tend to increase FIO₂ to draw SpO₂ up, but if SpO₂ is above the Target SpO₂, it will tend to draw FIO₂ down to draw SpO₂ down. Furthermore, the magnitude of the proportional gain term may be proportional to the distance, or value difference, of the SpO₂ from the Target SpO₂. As such, the further the current SpO₂ is from the Target SpO₂, the greater the proportional gain term will be. Still further, in some embodiments, if SpO₂ is outside of a certain range that includes the target SpO₂, then the proportional gain term may be greater. Overall, the proportional gain term may operate to create a tendency to affect the FIO₂ adjustment so as to draw the patient's SpO₂ toward the Target SpO₂, and the magnitude of the proportional gain term may be greater when the SpO₂ is further from the Target SpO₂. The proportional gain term may be unaffected by whether SpO₂ is increasing or decreasing. In the example method 400, a response time coefficient (K₁) is used in the determination of the proportional gain term. A higher response time coefficient is generally associated with a shorter patient response time. Furthermore, a higher response time coefficient yields a proportional gain term with a greater magnitude, which will cause the FIO₂ to increase (or decrease) at a faster rate.

The derivative gain term may operate as follows. When SpO₂ is evidenced as decreasing (as may be indicated by the current SpO₂ as compared with the last SpO₂, or an average of a set of most recent SpO₂ measurements compared to an average of a set of SpO₂ measurements prior to that, for example, or in other ways), the derivative gain term may operate to create a tendency to draw the FIO₂ up to correspondingly draw SpO₂ up. Furthermore, the magnitude of the derivative gain term may effectively be proportional to the effective rate of decrease of the SpO₂, in that it may be proportional to a difference between a current SpO₂ (or moving average of most recent SpO₂, for example) and a last SpO₂ (or a moving average of SpO₂ prior to the most recent SpO₂, for example). However, when SpO₂ is increasing, the derivative gain term may be 0, creating no tendency to draw the FIO₂ down to draw the SpO₂ down. In this sense, the derivative gain term may be considered to have an asymmetric effect on FIO₂ change, tending to increase FIO₂ when SpO₂ is decreasing, but having no symmetric effect of tending to decrease the FIO₂ when SpO₂ is increasing. The derivative gain term may be unaffected by the current SpO₂, as such, and based only on whether SpO₂ is increasing or decreasing, and, if decreasing, at what effective rate.

If SpO₂ is far above target and decreasing slowly, then the proportional gain term in the calculated FIO₂ correction may be negative (tending to draw FIO₂ down) and the derivative gain term may be positive (tending to draw FIO₂ up), but the proportional gain term may be greater in magnitude than the derivative gain term. In such an instance, the overall correction may be negative (so that FIO₂ is decreased) due to the proportional gain term, but the derivative gain term may effectively operate as a partial brake on, or to mitigate the magnitude of, the overall decrease.

However, if SpO₂ is slightly above target but decreasing rapidly, then the proportional gain term may be negative and the derivative gain term may be positive, but the proportional gain term may be lesser in magnitude than the derivative gain term. In such an instance, the overall FIO₂ adjustment may be positive due to the derivative gain term, but the proportional gain term may effectively operate as a partial brake on, or to mitigate the magnitude of, the overall increase. As such, in some embodiments, when SpO₂ is increasing, the FIO₂ correction may be entirely due to the proportional gain term, and FIO₂ will change so as to draw the SpO₂ towards the Target SpO₂. However, when the SpO₂ is decreasing, the proportional gain term and the derivative gain term may both operate to affect the FIO₂ adjustment, whether in an additive or opposite fashion.

It is notable that, in a rapidly decreasing situation, where, for example, patient SpO₂ is much lower than the target and decreasing rapidly, both the proportional gain term and the derivative gain term will be large and positive, leading to the FIO₂ being adjusted by a large increase. In this instance, the proportional gain term and the derivative gain term both contribute to an appropriate rapid increase in FIO₂ in response to what may be an urgent actual or approaching patient desaturation crisis.

In FIG. 4A, at operation 402, the system waits for a predetermined time increment, such as one second, (however, in some embodiments, the wait loop time increment may be different, e.g., a fraction of a second, between 1-2 seconds, multiple seconds, more than one minute, an irregular or varying interval, etc.). At operation 404, patient SpO₂ (e.g., a current SpO₂) is determined. For example, an oximetry sensor, coupled with the patient, may be used to obtain signals representative of the patient's SpO₂, and a controller may receive the signals and determine the patient's SpO₂ based on the signals. At operation 406, the system confirms that the determined SpO₂ is within a valid range, such as by confirming that the SpO₂ is between 0% and 100%. In some embodiments, more than just a single measured SpO₂ may be used, such as, for example, an SpO₂ moving average, a noise filtered SpO₂ value, etc. Once patient SpO₂ has been determined, both the proportional gain term and derivative gain term can be determined in any order or simultaneously. The left branch of method 400 following operation 406 provides operations for determining the proportional gain term and the right branch of method 400 following operation 406 provides operations for determining the derivative gain term, according to some embodiments. The operations within each branch may be performed in any order. The left branch for determining the proportional gain term will be described first.

At operation 408, the system calculates an SpO₂ error term as the difference between the Target SpO₂ and the determined SpO₂ from operation 404. Accordingly, the SpO₂ error term is larger in magnitude when the patient's SpO₂ is further from the Target SpO₂. If the patient's SpO₂ is below the Target SpO₂, the SpO₂ error term will be positive to cause a respective increase in the FIO₂ level. If the patient's SpO₂ is above the Target SpO₂, the SpO₂ error term will be negative to cause a respective decrease in the FIO₂ level.

At operation 410, the response time coefficient K₁ is determined or obtained (if previously determined). As discussed above, the response time coefficient K₁ is a function of the patient response time. Generally, the response time coefficient K₁ may be inversely related to the patient response time, such that longer patient response times yield a smaller K₁ and shorter patient response times yield a larger K₁. In other words, patients with fast SpO₂ response times may have a larger response time coefficient K₁ to boost the rate that the FIO₂ level is changed.

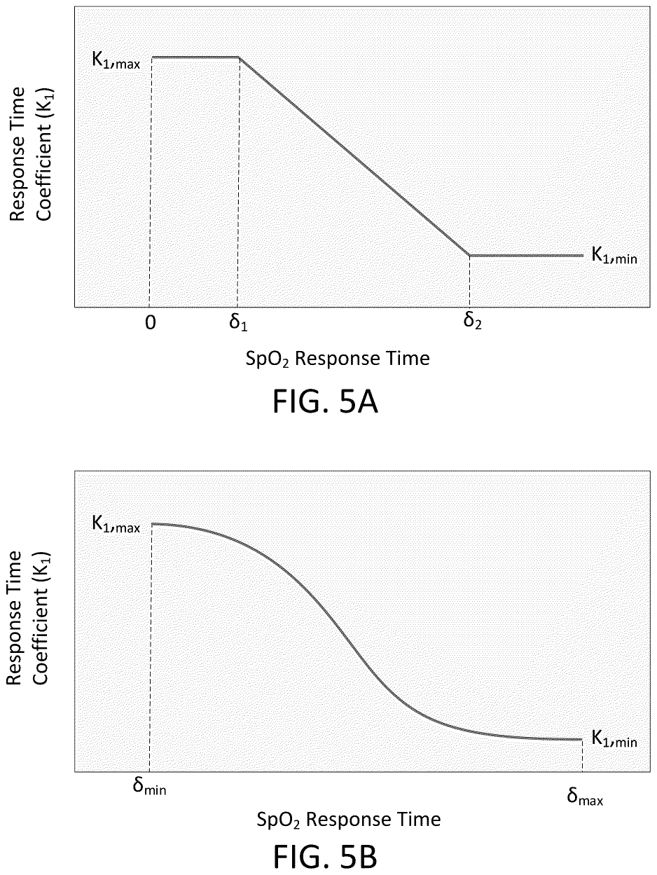

FIGS. 5A and 5B illustrate example simulations for determining the response time coefficient K₁ as a function of the SpO₂ response time. In FIG. 5A, K₁ may be set to a max value for any SpO₂ response time below a first threshold (δ₁) and set to a min value for any SpO₂ response time above a second threshold (δ₂). In an example, the max value of K₁ may be 4 and the min value of K₁ may be 1. In general, K₁ may range between any two values, such as a max value of 10 and a min value of 0. According to an embodiment, the K₁ value may be determined by a linear relationship for SpO₂ response times between the first threshold δ₁ and the second threshold δ₂. In FIG. 5B, K₁ may have a sigmoidal relationship with the SpO₂ response time between a min SpO₂ response time (δₘᵢₙ) and a max SpO₂ response time (δₘₐₓ). Any other functional relationships may be considered between K₁ and SpO₂ response time, such as a logarithmic relationship, polynomial relationship, exponential relationship, etc.

Returning to method 400, at operation 412 an SpO₂ gain coefficient (K₂) is determined based at least in part on the patient's current SpO₂ level. According to some embodiments, if the SpO₂ level is within a particular range (e.g., between 93% and 99%), then K₂ may be set to a first smaller value (K_{2,min}), and if the SpO₂ level is outside of the particular range, then K₂ may be set to a second larger value (K_{2,max}). The SpO₂ gain coefficient can lead to a much more rapid FIO₂ response when patient SpO₂ is far from a target value such as 94%, which can include a much more rapid FIO₂ increase when patient SpO₂ is very low. In some embodiments, however, proportional gain is not varied based on any threshold or range.

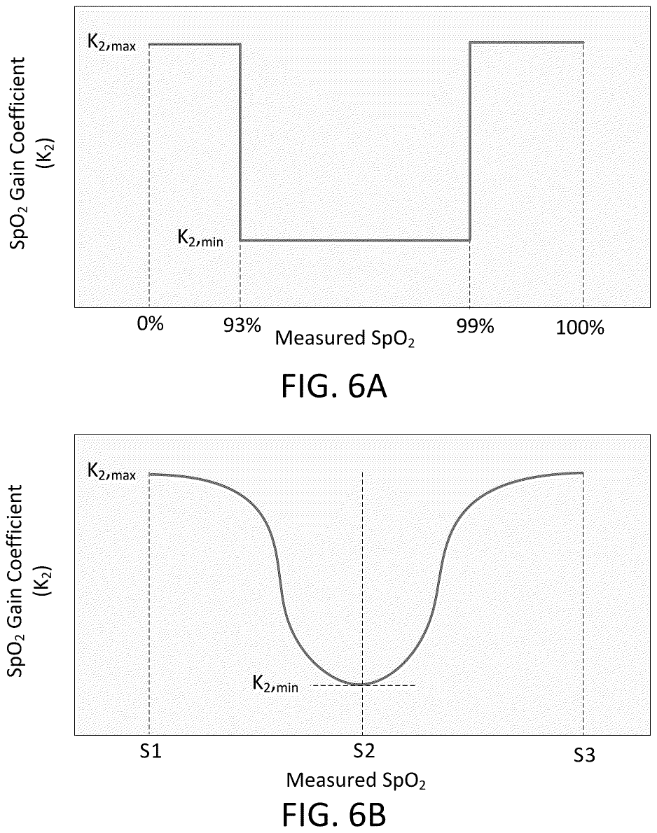

In some embodiments, K_{2,min} may be, for example, 0.0025 (or, e.g., between 0.001 and 0.015, such as between 0.001 and 0.005, between 0.005 and 0.010, or between 0.010 and 0.015), and K_{2,max} may be, for example, 0.005, (or, e.g., between 0.002 and 0.03, such as between 0.002 and 0.01, between 0.01 and 0.02, or between 0.02 and 0.03). The value of K_{2,max} may be double the value of K_{2,min}. FIG. 6A illustrates an example of how K₂ may change between two values (K_{2,max} and K_{2,min}) depending on whether the measured SpO₂ is within a given range or is within a range around a target SpO₂ value (e.g., +/−2% around a target SpO₂ level). In the example shown in FIG. 6A, the given range is between 93% and 99%.

In some embodiments, the value of K₂ is a function of the patient's measured SpO₂ level. FIG. 6B illustrates an example of how K₂ may be some sigmoidal function of the measured SpO₂. In this example, the value of K₂ decreases at some sigmoidal rate between a first SpO₂ level (S1) and a second SpO₂ level (S2), and increases at some sigmoidal rate between the second SpO₂ level (S2) and a third SpO₂ level (S3), thus reaching a minimum value (K_{2,min}) at the second SpO₂ level (S2). The first and third SpO₂ levels (S1 and S3) may correspond to the set SpO₂ range discussed above (e.g., 93% and 99%). According to some embodiments, K₂=K_{2,max} for an SpO₂ measurement less than S1 or greater than S3.

Returning to method 400, at operation 414 a proportional gain coefficient (Kₚ) is determined based on a function of both the response time coefficient K₁ and the SpO₂ gain coefficient K₂. In some examples, Kₚ is the product of K₁ and K₂. In some examples, Kₚ is the sum of K₁ and K₂. Any expression involving K₁ and K₂ could be considered for determining Kₚ. According to some embodiments, the value of Kₚ increases for both increasing values of K₁ and increasing values of K₂.

Looking back at the right branch of method 400 following operation 406, at operation 416 the system calculates a derivative error (DE) based on how quickly the patient's SpO₂ level has changed over a period of time. According to some embodiments, the derivative error is equal to a difference between a LastError term and an Error term, where Error=average of (Target SpO₂−Measured SpO₂) for N most recent times, and LastError=average of (Target SpO₂−Measured SpO₂) for N next most recent times. Conceptually, for a constant Target SpO₂, DE will be negative (which will produce a positive Derivative Gain term, as explained below), when Error is greater than LastError−that is, when SpO₂ is, overall, decreasing. Conversely, DE will be positive (which may produce a Derivative Gain term of 0, as explained below), when LastError is greater than Error−that is, when SpO₂ is, overall, increasing.

In some embodiments, moving averages are utilized, such as to take into account different SpO₂ over different amounts of time or samples, or to effectively filter out random or unpredictable variation or noise, or to prevent undesirably rapidly varying or choppy FIO₂ or SpO₂ response, for example. However, in some embodiments, other methods are used, including methods that use only the current and last SpO₂ measurements, or that use a different technique in determining an effective rate of increase or decrease in SpO₂, or that use a different technique in filtering or smoothing, for example.

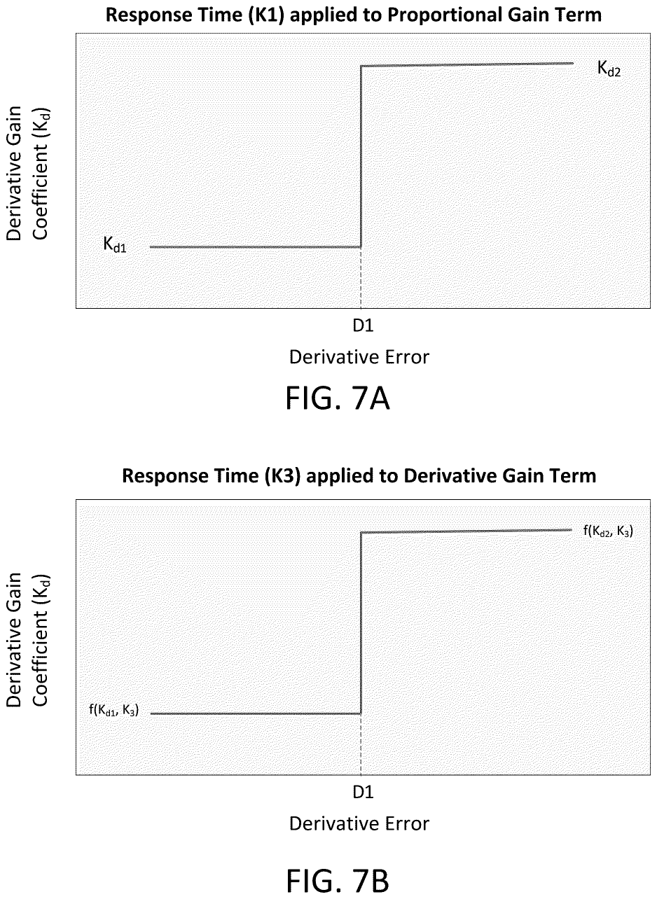

At operation 418, if DE is equal to or less than 0, then the derivative gain coefficient (K_{d}) is set to a first value, whereas, if DE is greater than 0, then K_{d} is set to a second value. In some examples, the first value is a negative fraction or negative whole number and the second value is 0 (effectively setting the derivative gain term to 0 in situations where the patient's SpO₂ is rising towards the Target SpO₂). In some examples, the second value can be a value other than 0. FIG. 7A illustrates how the derivative gain coefficient K_{d} may be determined based on the derivative error (DE). For a DE less than a threshold value (D1), K_{d} may be set to the first value (K_{d1}) and for a DE greater than the threshold value D1, K_{d} may be set to the second value (K_{d2}). In an example, D1=0, K_{d1}=−0.07, and K_{d2}=0. In other examples, any of the values for D1, K_{d1}, and K_{d2} can be different depending on the application. For example, K_{d1} may range between −0.01 and −1.0, and K_{d2} may range between 0 and 1.

At operation 420, an FIO₂ correction value (FC) is determined based on a function of the proportional gain term and the derivative gain term. According to some embodiments, the proportional gain term is equal to the product of the SpO₂ error term and the proportional gain coefficient Kₚ, and the derivative gain term is equal to the product of the derivative error (DE) and the derivative gain coefficient K_{d}. In this example, the proportional gain coefficient Kₚ is based, at least in part, on the value of the response time coefficient K₁, which is a function of the measured patient response time.

At operation 422, an updated FIO₂ level is set on the ventilator. The updated FIO₂ level may be determined as the Current FIO₂ level+FC. In this way, the FIO₂ level is adjusted either positively or negatively depending on the value and sign of the FIO₂ correction value In some embodiments, FC is not applied if the current FIO₂ level would be brought out of a valid range (e.g., if the current FIO₂ level would be raised to a level greater than 100% or lowered to a level less than some lower threshold, such as 20%). The FIO₂ setting on the ventilator may be adjusted via, for example, actuation of an oxygen source valve on the ventilator with a corresponding adjustment to the air flow source, which could be a high-pressure source controlled with a valve or a compressor or high-flow blower. Generally, such adjusting can include, as appropriate, the FIO₂ setting being increased, decreased, or kept the same if the FIO₂ correction (FC) is equal to 0 so that the updated FIO₂ is the same as the current FIO₂.

Following operation 422, method 400 returns to operation 402 to continue physiologic closed-loop control of the FIO₂ level delivered to the patient. In some embodiments, this physiologic closed-loop control process continues for as long as the ventilator remains active on the patient. In some embodiments, this physiologic closed-loop control process continues until the ventilator is shut off, or is paused by some fault or failure that triggers a fallback to nonoperating alarms state or open-loop operation where parameters are static unless adjusted by the user, or until the physiologic closed-loop control process is manually halted via a user interface on the ventilator. In some embodiments, this closed loop control process may be interrupted (e.g., automatically paused) at any time to enter a patient response time procedure for determining the patient response time. Further details regarding an example patient response time procedure are provided with reference to FIGS. 9 and 10.

FIG. 4B illustrates another method 401 of a physiologic closed-loop control process for adjusting the FIO₂ level delivered to a patient. Method 400 includes many similar operations to method 400. However, in method 401, the patient response time coefficient K₁ is instead applied to the derivative gain term instead of the proportional gain term. In this example, a shorter patient response time yields a higher derivative gain term, which causes the FIO₂ level to increase faster when the patient's SpO₂ level is decreasing more quickly.

Method 401 begins with the same operations 402, 404, and 406 as described above for method 400. The SpO₂ error term is determined in the same way as discussed above in operation 408 of the proportional gain branch, and the derivative error (DE) is also determined in the same way as discussed above in operation 416.

At operation 424 of the proportional gain branch, the proportional gain coefficient Kₚ is set to either a first value (K_{p,min}) or a second value (K_{p,max}) depending on whether SpO₂ is within a set range. This is similar to how the SpO₂ gain coefficient K₂ was determined in operation 412 of method 400. Note that in method 401, the proportional gain coefficient Kₚ is not based on the patient response time, in contrast to method 400.

At operation 425 of the derivative gain branch, a derivative gain parameter (DG) is set to a first value if the derivative error is less than or equal to 0, and DG is set to a second value if the derivative error is greater than 0. This is similar to how the derivative gain coefficient K_{d} was determined in operation 418 of method 400. However, in method 401, the derivative gain coefficient K_{d} is based on both the value of the derivative gain parameter DG and on the value of the response time coefficient K₃. Note that response time coefficient K₃ may have the same value as response time coefficient K₁, or if may have a different value.

At operation 426, the response time coefficient K₃ is determined or accessed (if previously determined) in the same manner as discussed above for operation 410 of method 400. Note that response time coefficient K₃ may have the same value as response time coefficient K₁. In some embodiments, K₃ is different from K₁ and may be determined based on a different function of the SpO₂ response time (e.g., different from the function as shown in FIGS. 5A and 5B for K₁). In some examples, K₃ may be determined using a similar function as K₁ (as illustrated in FIGS. 5A and 5B), but with different values for K_{1,max} and K_{1,min}.

At operation 428, the derivative gain coefficient K_{d} is determined as being a function of both the derivative gain parameter DG (e.g., being either a first value K_{d1} or a second value K_{d2}) and the response time coefficient K₃. In some examples, K_{d} is the product of DG and K₃. In some examples, K_{d} is the sum of DG and K₃. Any expression involving DG and K₃ could be considered for determining K_{d}. According to some embodiments, the value of K_{d} increases for an increasing value of DG and increasing values of K₃. FIG. 7B illustrates how the derivative gain coefficient K_{d} may be determined based on the derivative error (DE) while also taking into account the patient response time. For a DE less than a threshold value (D1), K_{d} may be set to a value that is a function of the first value K_{d1} and the response time coefficient K₃ and for a DE greater than the threshold value D1, K_{d} may be set to a value that is a function of the second value K_{d2} and the response time coefficient K₃. In an example, D1=0, K_{d1}=−0.07, and K_{d2}=0. In other examples, any of the values for D1, K_{d1}, and K_{d2} can be different depending on the application. For example, K_{d1} may range between −0.01 and −1.0, and K_{d2} may range between 0 and 1.

At operation 430, an FIO₂ correction value (FC) is determined based on a function of the proportional gain term and the derivative gain term. Just as discussed above for method 400, the proportional gain term is equal to the product of the SpO₂ error term and the proportional gain coefficient Kₚ, and the derivative gain term is equal to the product of the derivative error DE and the derivative gain coefficient K_{d}. In this example, the derivative gain coefficient K_{d} is based, at least in part, on the value of the response time coefficient K₃, which is a function of the measured patient response time. An updated FIO₂ level is set on the ventilator based on the determined FIO₂ correction value (FC) at operation 422, in the same fashion as described above in method 400.

FIG. 4C illustrates another method 403 of a physiologic closed-loop control process for adjusting the FIO₂ level delivered to a patient. Method 403 includes many similar operations to both method 400 and method 401. According to some embodiments, the patient response time is factored into the determination of both the proportional gain term and the derivative gain term. Accordingly, the proportional gain branch is similar to that described in method 400 and the derivative gain branch is similar to that described in method 401. In this example, a faster patient response time yields both a higher proportional gain term and a higher derivative gain term.

At the conclusion of both the proportional gain branch and the derivative gain branch of method 403, the FIO₂ correction value (FC) is determined based on a function of the proportional gain term and the derivative gain term at operation 432. Just as discussed above for methods 400 and 401, the proportional gain term is equal to the product of the SpO₂ error term and the proportional gain coefficient Kₚ, and the derivative gain term is equal to the product of the derivative error DE and the derivative gain coefficient K_{d}. In this example, the proportional gain coefficient Kₚ is based, at least in part, on the value of the response time coefficient K₁ and the proportional gain coefficient K₂, and the derivative gain coefficient K_{d} is based, at least in part, on the value of the response time coefficient K₃. Both K₁ and K₃ are functions of the measured patient response time. An updated FIO₂ level is set on the ventilator based on the determined FIO₂ correction value (FC) at operation 422, in the same fashion as described above in method 400.

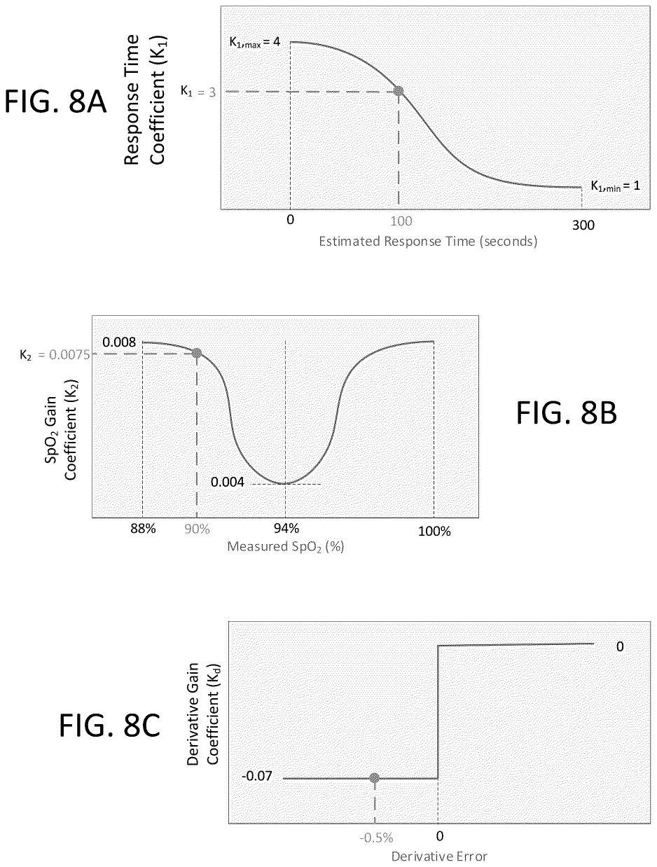

An example will now be described for a hypothetical patient using the simulated graphs shown in FIGS. 8A-8C to determine various parameters used in the physiologic closed-loop control method 400. A response time procedure may be activated to estimate the patient's response time as being 100 seconds (more detail on how this is performed is provided with reference to FIGS. 9 and 10). Following method 400, the patient's SpO₂ level is measured to be 90% at operation 404. In this example scenario, the patient's Target SpO₂ level is 94%, so this patient is below their target and needs their SpO₂ level increased. Furthermore, at operation 408, the SpO₂ Error is 94%−90%=4%.

At operation 410, the response time coefficient K₁ is determined based on a function of the patient response time. FIG. 8A shows an example relationship between the patient response time and the response time coefficient K₁. In this example, a patient response time of 100 seconds yields a response time coefficient K₁=3. In this example, the response time coefficient K₁ ranges between 1 and 4, but in other examples, the response time coefficient K₁ can range between any values.

At operation 412, the SpO₂ gain coefficient K₂ is determined based on a function of the measured SpO₂ level. FIG. 8B shows an example relationship between the measured SpO₂ level and the SpO₂ gain coefficient K₂. In this example, the patient's measured SpO₂ level of 90% yields an SpO₂ gain coefficient K₂ of 0.0075. In this example, the SpO₂ gain coefficient K₂ ranges between 0.008 and 0.004, but in other examples, the SpO₂ gain coefficient K₂ can range between any values.

At operation 414, the proportional gain coefficient Kₚ is determined as a function of both K₁ and K₂. In some embodiments, Kₚ is equal to K₁*K₂, which in this example, means that Kₚ=3*0.0075=0.0225.

At operation 416, the derivative error (DE) is determined based on how the patient's SpO₂ level has changed over time. In this example, the patient's SpO₂ level has been falling over the last several cycles which has yielded a derivative error (DE) of −0.5%.

At operation 418, the derivative gain coefficient K_{d} is determined based on DE. FIG. 8C shows an example relationship between the determined derivative error (DE) and the derivative gain coefficient K_{d}. In this example, the derivative error was determined to be −0.5%, which is less than 0, and thus yields a derivative gain coefficient K_{d} of −0.07. In this example, the derivative gain coefficient K_{d} is set to either −0.07 or 0, but in other examples, the derivative gain coefficient K_{d} can be set to any values based on the derivative error (DE).

At operation 420, the FIO₂ correction (FC) is determined as (Kₚ*SpO₂ error)+(K_{d}*DE)=(0.0225*4%)+(−0.07*−0.5%)=0.125%. Accordingly, at operation 422, the FIO₂ level delivered to this patient will be increased by 0.125% from its current level. The control process 400 then returns back to operation 402 to continue the process of monitoring the patient's SpO₂ level and adjusting the FIO₂ level, if needed.

As discussed above, the ventilator may be configured to enter a response time procedure at any time to determine a patient's SpO₂ response time. In some examples, a response time procedure is initiated before the ventilator enters the active period (e.g., FIO₂ physiologic closed-loop control). In some examples, the FIO₂ physiologic closed-loop control is paused at any time to initiate the response time procedure and determine the patient's SpO₂ response time before returning to the FIO₂ physiologic closed-loop control of the active period.

FIG. 9 illustrates an example process flow of the response time procedure, according to an embodiment. At a first operation 902 of the response time procedure, the FIO₂ level is held at a first value while the SpO₂ is monitored to ensure that it has reached a steady state before proceeding to the next operation. According to some embodiments, the SpO₂ level is considered to have reached a steady state when it does not change beyond a given percentage over a given period of time. For example, the SpO₂ level may reach a steady state when it does not change more than 1% over a one minute period. The SpO₂ level may have reached a steady state if a derivative of the SpO₂ level is less than some threshold value (e.g., exhibiting little variation over time).

Once the SpO₂ level has reached a steady state, the response time procedure continues to operation 904 where a step increase is made to the FIO₂ level to bring it from the first level (FIO_{2,low}), which is the patient's current FIO₂, to a second level (FIO₂,high). This change in the FIO₂ level during the response time procedure may also be referred to as a test change in the FIO₂. FIO₂ may be increased by any given amount. In some examples, FIO₂ is increased by 10%, 15%, 20%, 25%, 30%, 40%, or 50%. In some examples, FIO₂ is increased up to a fixed percentage, such as 70%, 75%, 80%, 85%, or 90%. In some examples, FIO₂ is increased by a proportion of the difference between the current FIO₂ level and 100% FIO₂ (e.g., if current FIO₂=40%, the level may be increased by half of the difference between 40% and 100%, which would 40%+(0.5*60%)=70%). In contrast to the gradual change of FIO₂ during the active period, this increase to the FIO₂ setting on the ventilator may be instant. At operation 906, the patient's SpO₂ level is monitored over a first time period (tₛₜₑₚ₁), which may be equal to the time period that the FIO₂ level is held at FIO_{2,high}. During this time, the oxygen saturation in the patient's blood is expected to rise as a result of the sharp increase in FIO₂. The rate at which the patient's blood oxygen saturation rises is determined by the rate at which their measured SpO₂ level rises. Their response time may then be determined based on this rate as discussed in more detail herein.

At operation 908, the FIO₂ level is stepped back down to the first level (FIO_{2,low}). This change in the FIO₂ level during the response time procedure may also be referred to as a test change in the FIO₂. In some embodiments, the transition to operation 908 occurs after determining that the patient's SpO₂ level has reached a steady state. In some embodiments, the transition to operation 908 occurs after a set period of time (e.g., a predetermined tₛₜₑₚ₁). In either case, the decrease to the FIO₂ setting on the ventilator may be instant. In some examples, the FIO₂ level does not return to the same first level (FIO_{2,low}), but to any different level. At operation 910, the patient's SpO₂ level is monitored over a second time period (tₛₜₑₚ₂), which may be equal to the time period that the FIO₂ level is held at FIO₂,low. During this time, the patient's blood oxygen saturation is expected to drop as a result of the sharp decrease in FIO₂. The rate at which the patient's blood oxygen content drops is determined by the rate at which their measured SpO₂ level decreases.

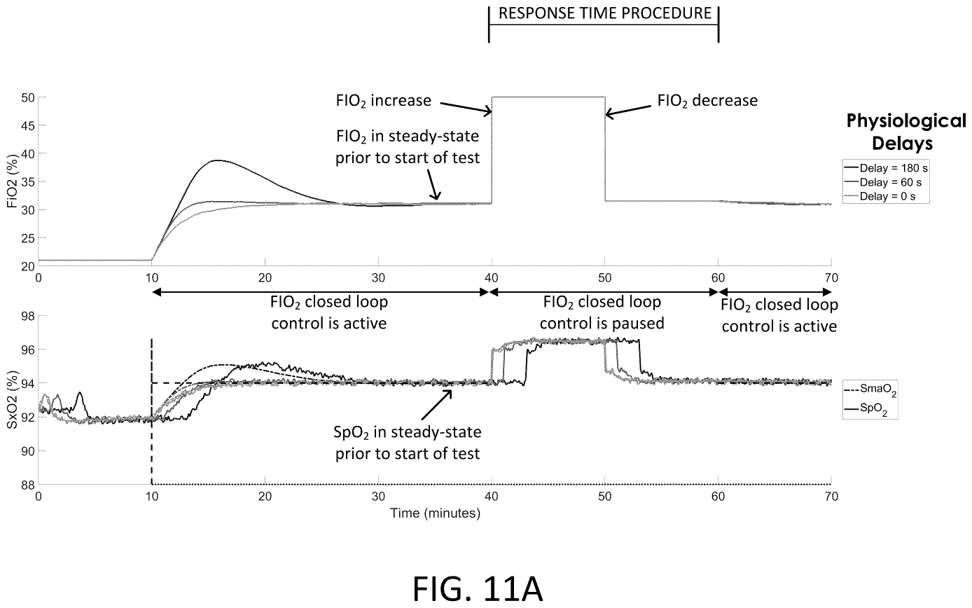

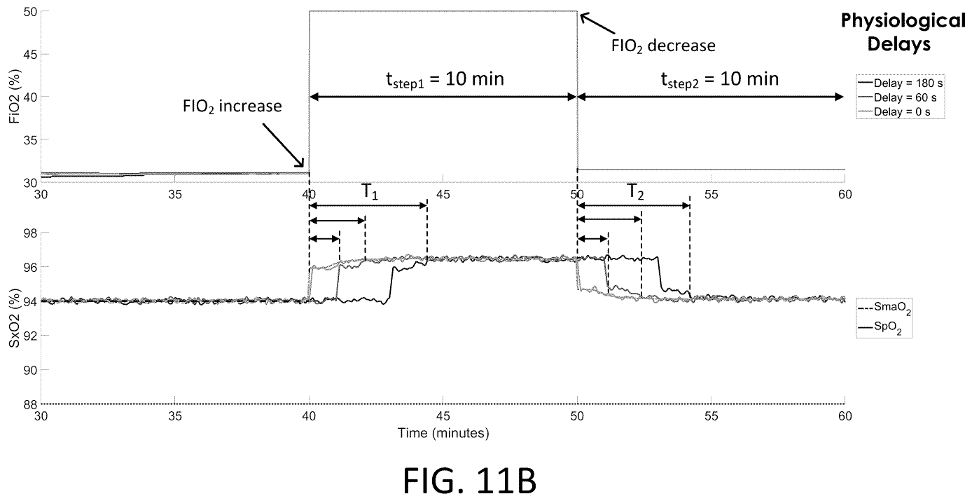

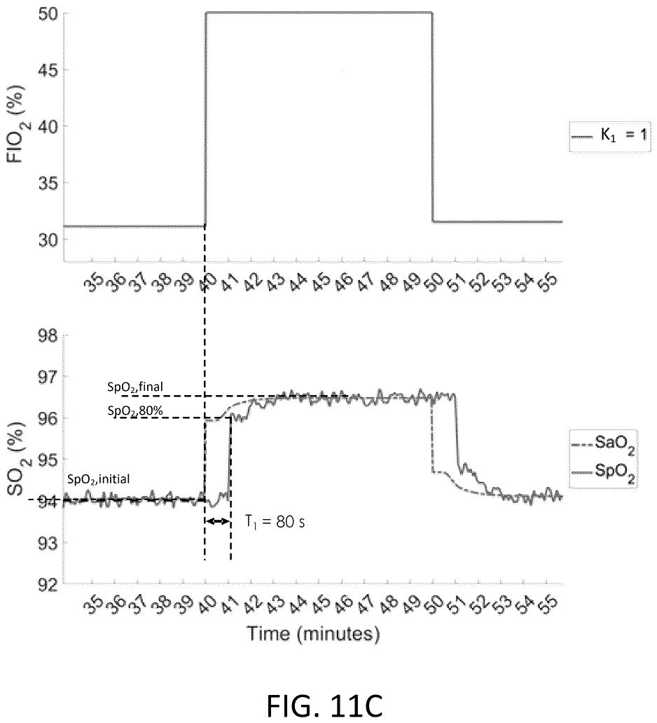

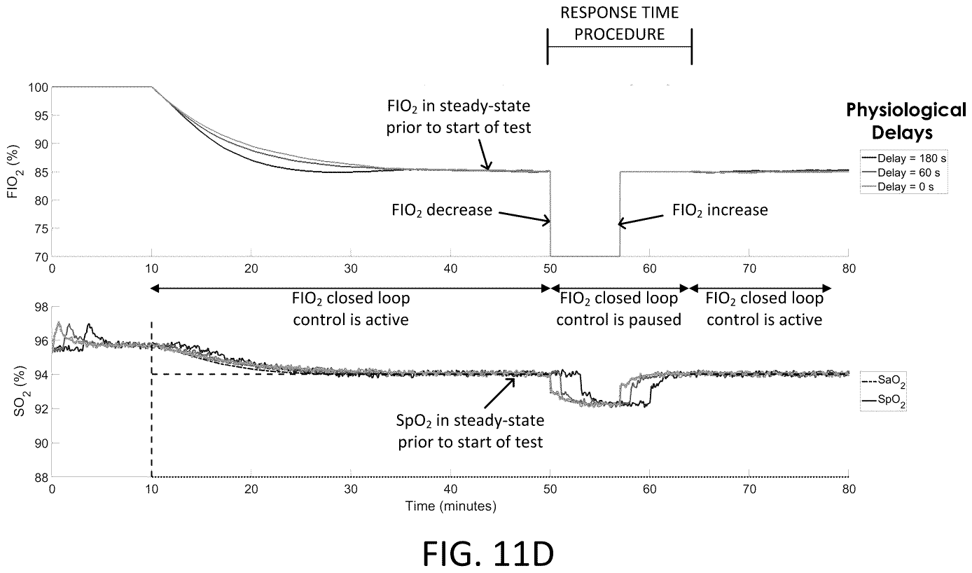

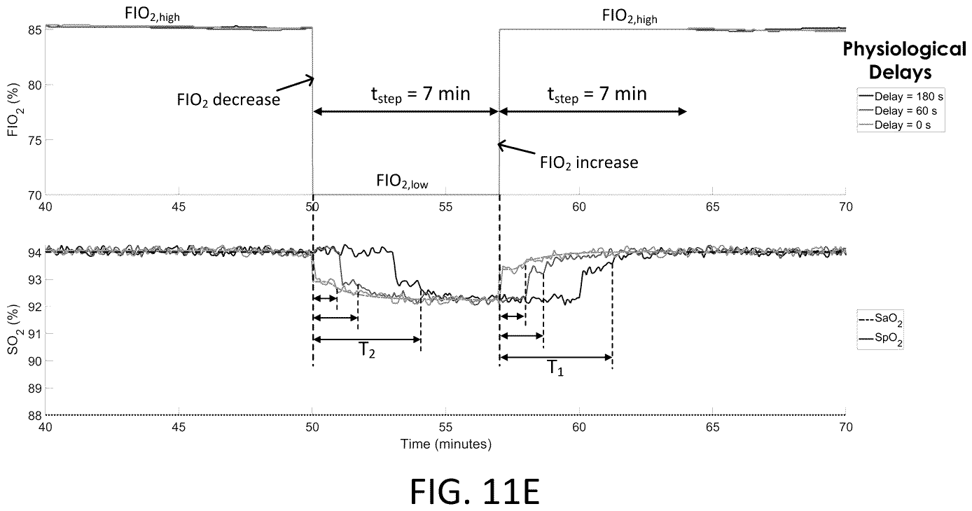

At operation 912, the patient response time is determined for the given patient. In some embodiments, the transition to operation 912 occurs after determining that the patient's SpO₂ level has reached a steady state. In some embodiments, the transition to operation 912 occurs after a set period of time (e.g., a predetermined tₛₜₑₚ₂). In either case, there are several possible ways to determine a patient response time given the monitored changes in the patient's SpO₂ level over each of tₛₜₑₚ₁ and tₛₜₑₚ₂. In some examples, the patient response time may be related to a time that it takes for the SpO₂ level to reach steady state during each of tₛₜₑₚ₁ and tₛₜₑₚ₂. In some examples, the patient response time may be related to a time that it takes for the SpO₂ level to reach a percentage of its final steady state value. These examples are discussed in more detail with reference to FIGS. 11A-11C.

It should be noted that in the example response time procedure of FIG. 9, the FIO₂ level is first increased and then (following a time of tₛₜₑₚ₁) decreased to monitor the effect on SpO₂. However, the same process may be performed by first decreasing the FIO₂ level and then (following a time of tₛₜₑₚ₁) increasing the FIO₂ level.

FIG. 10 illustrates a more detailed method 1000 of the response time procedure discussed in reference to FIG. 9. This discussion is followed by further discussion of simulated SpO₂ data during the response time procedure for patients with different response times.

At operation 1002, a determination is made regarding certain criteria before proceeding with any changes to the FIO₂ setting of the ventilator. For example, both SpO₂ and FIO₂ may need to be in a steady state. As discussed above, steady state can be determined several different ways depending on the application, but generally is defined as exhibiting little to no change over a given period of time. In some examples, either SpO₂ or FIO₂ may be considered to be in steady state if their value has not changed by more than 0.5%, more than 1%, or more than 2%, over a period of 1 minute, 3 minutes, or 5 minutes. Steady state may be determined by a mathematical calculation, expression or model, including one or more terms of one or more variables, or a function of one or more variables. The calculation, expression, model or function may take as input the patient's SpO₂ (or one or more other patient oxygenation parameters) or change in SpO₂ over a previous period of time, or previous times.

According to some embodiments, another criteria in operation 1002 may include determining that the current FIO₂ level is below a threshold FIO₂ level and similarly that a current SpO₂ level is below a threshold SpO₂ level. These determinations may be made in a situation where the FIO₂ level is first increased and then decreased during the response time procedure. If the FIO₂ level is currently at a value that is too high, there will not be enough room to increase it further and perform effective monitoring of the SpO₂ level (since the FIO₂ level cannot be increased over 100%). In a similar fashion, if the patient's SpO₂ level is currently at a value that is too high, there will not be enough room to effectively monitor its increase since it also can't increase over 100%. In an example, the threshold FIO₂ level may be 80% and the threshold SpO₂ level may be 96%.

It should be noted that the threshold determinations regarding FIO₂ and SpO₂ can also be made in another situation where the FIO₂ level is first decreased and then increased during the response time procedure. In this situation, if the FIO₂ level is currently at a value that is too low, there will not be enough room to decrease it further and perform effective monitoring of the SpO₂ level (since the FIO₂ level cannot be decreased to less than the oxygen content of the room air, which is typically around 21%). In a similar fashion, if the patient's SpO₂ level is currently at a value that is too low, there will not be enough room to effectively monitor its decrease since it also can't decrease under 0%.

According to some embodiments, the ventilator system may determine in operation 1002 whether to perform the test by using a step increase in FIO₂ or using a step decrease in FIO₂ based on the current SpO₂ level of the patient and/or the current FIO₂ level of the ventilator. For example, if the patient's current SpO₂ level is below a given amount (e.g., less than 96%) and the FIO₂ level is below a given amount (e.g., less than 80%), then the test may proceed by performing a step increase in FIO₂. However, if the patient's current SpO₂ level is above a given amount (e.g., greater than 85%) and the FIO₂ level is above a given amount (e.g., greater than 50%), then the test may proceed by performing a step decrease in FIO₂. In some examples, if the patient's current SpO₂ level and the ventilator's FIO₂ level are each within a middle range (e.g., SpO2 between 90% and 94% and FIO₂ between 50% and 70%), then either a step increase in FIO₂ or a step decrease in FIO₂ can be performed, or both a step increase in FIO₂ and a step decrease in FIO₂ can be performed.

Once the initial criteria have been met in operation 1002, method 1000 proceeds to operation 1004 where the FIO₂ setting of the ventilator is changed from a first FIO₂ level to a second FIO₂ level. This change in the FIO₂ level may also be referred to as a test change in the FIO₂. In some examples, the FIO₂ level is changed from the current FIO₂ level to a higher FIO₂ level (e.g., raised by 10%, by 20%, or by 30%). In some examples, the FIO₂ level is changed from the current FIO₂ level to a lower FIO₂ level (e.g., lowered by 10%, by 20%, or by 30%). The change may be performed instantly to result in a step change in the FIO₂ level.

At operation 1006, the patient's SpO₂ level is monitored using, for example, a pulse oximeter or other similar sensor. At operation 1008, a determination is made whether the patient's SpO₂ has reached a first steady state. According to some embodiments, a short pause or delay may exist between operation 1006 and operation 1008 to allow for some time before the patient's blood oxygen saturation shows any response to the change in the FIO₂ level. In some embodiments, the first steady state value must be at an SpO₂ level that is different than the current SpO₂ level.

If the SpO₂ has not yet reached the first steady state value (e.g., it is found to still be increasing, decreasing, has not changed from its current level, or changed in the opposite direction than the change in FIO₂ setting), or if a fixed period of time has not yet passed, then method 1000 returns to operation 1006 to continue monitoring the patient's SpO₂. However, if it is determined in operation 1008 that the SpO₂ level has reached a steady state (or if the fixed period of time has passed), method 1000 continues to operation 1010 where a first time parameter (T₁) is determined and/or recorded. In some embodiments, if the SpO₂ level cannot reach steady state after a certain period of time has passed (e.g., after 10 minutes), then method 1000 may terminate and issue an error message or statement that the test could not be successfully completed.

The first time parameter (T₁) may correspond to a time that it takes to reach a given change in the monitored SpO₂ level during its increase or decrease towards the first steady state value. In some examples, T₁ corresponds to the time it takes for the SpO₂ level to reach a given percentage (e.g., 70%, 80%, 90%, or 100%) of the total change in SpO₂ from its initial level to its final level at the first steady state value.

Upon determination of T₁, method 1000 proceeds to operation 1012 where the FIO₂ setting on the ventilator is changed from the second FIO₂ level to a third FIO₂ level. This change in the FIO₂ level may also be referred to as a test change in the FIO₂. In examples where the FIO₂ level was increased in operation 1004, the third FIO₂ level may be lower than the second FIO₂ level, such that a step decrease is performed in the FIO₂ level in operation 1012. In examples where the FIO₂ level was decreased in operation 1004, the third FIO₂ level may be higher than the second FIO₂ level, such that a step increase is performed in the FIO₂ level in operation 1012. In some embodiments, the second FIO₂ level may be substantially equal to the “current” FIO₂ level at the conclusion of operation 1002, such that the change in FIO₂ up through operation 1012 resembles a square wave.

At operation 1014, the patient's SpO₂ level is monitored using, for example, a pulse oximeter or other similar sensor. At operation 1016, a determination is made whether the patient's SpO₂ has reached a second steady state value. According to some embodiments, a short pause or delay may exist between operation 1014 and operation 1016 to allow for some time before the patient's blood oxygen saturation shows any response to the change in the FIO₂ level. In some embodiments, the second steady state value must be at an SpO₂ level that is different than the first steady state SpO₂ level. If the SpO₂ has not yet reached steady state (e.g., it is found to still be increasing or decreasing, has not changed from its current level, or changed in the opposite direction than the change in FIO₂ setting), then method 1000 returns to operation 1014 to continue monitoring the patient's SpO₂. However, if it is determined in operation 1016 that the SpO₂ level has reached a steady state, method 1000 continues to operation 1018 where a second time parameter (T₂) is determined and/or recorded. In some embodiments, if the SpO₂ level cannot reach steady state after a certain period of time has passed (e.g., after 10 minutes), then method 1000 may terminate and issue an error message or statement that the test could not be successfully completed.

Like T₁, the second time parameter (T₂) may correspond to a time that it takes to reach a given change in the monitored SpO₂ level during its increase or decrease towards the second steady state. In some examples, T₂ corresponds to the time it takes for the SpO₂ level to reach a given percentage (e.g., 70%, 80%, 90%, or 100%) of the total change in SpO₂ from its initial level (at the first steady state) to its final level at the second steady state.

At operation 1020, the patient response time is determined based on a function of T₁ and T₂ or is based on a function of at least one of T₁ or T₂. In some examples, the patient response time is provided as the minimum between T₁ and T₂, the maximum between T₁ and T₂, or the mean between T₁ and T₂. Any other functions involving T₁ and T₂ could be used as well in determining a patient response time.

At operation 1022, the ventilator system returns to the active period and continues the FIO₂ physiologic closed loop control. As noted above, the FIO₂ physiologic closed loop control may be paused at any time to execute the response time procedure. According to some embodiments, the FIO₂ physiologic closed loop control process uses the patient response time determined in operation 1020 in order to affect the adjustment to the delivered FIO₂ level, as discussed in more detail with reference to FIGS. 4A-4C.

FIGS. 11A-11E illustrate simulated SpO₂ measurements from different patients during the execution of an example response time procedure on the ventilator that uses a relatively long (e.g., greater than 1 minute) step change in FIO₂ to allow the patient's SpO₂ to reach a steady state. In FIGS. 11A-11C, the FIO₂ level is first increased followed by a decrease to respectively monitor the way in which the patient's SpO₂ level increases to a steady state and then decreases. In FIGS. 11D and 11E the FIO₂ level is first decreased followed by an increase to respectively monitor the way in which the patient's SpO₂ level decreases to a steady state and then increases.

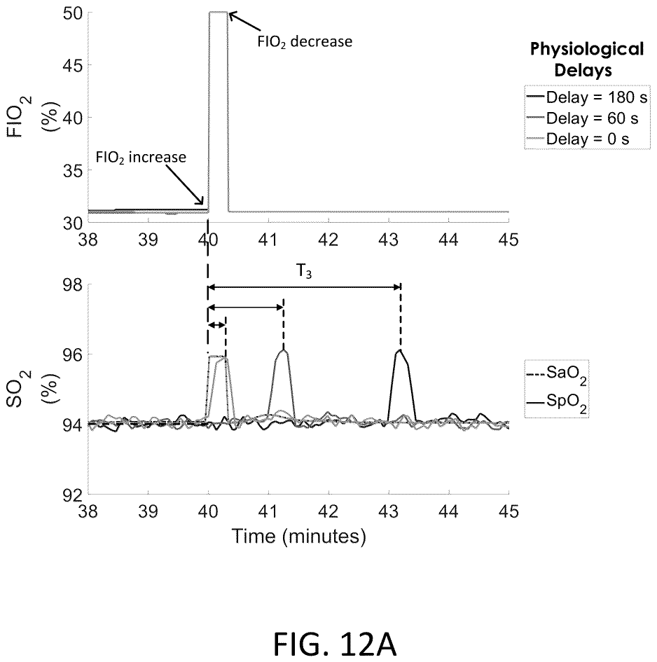

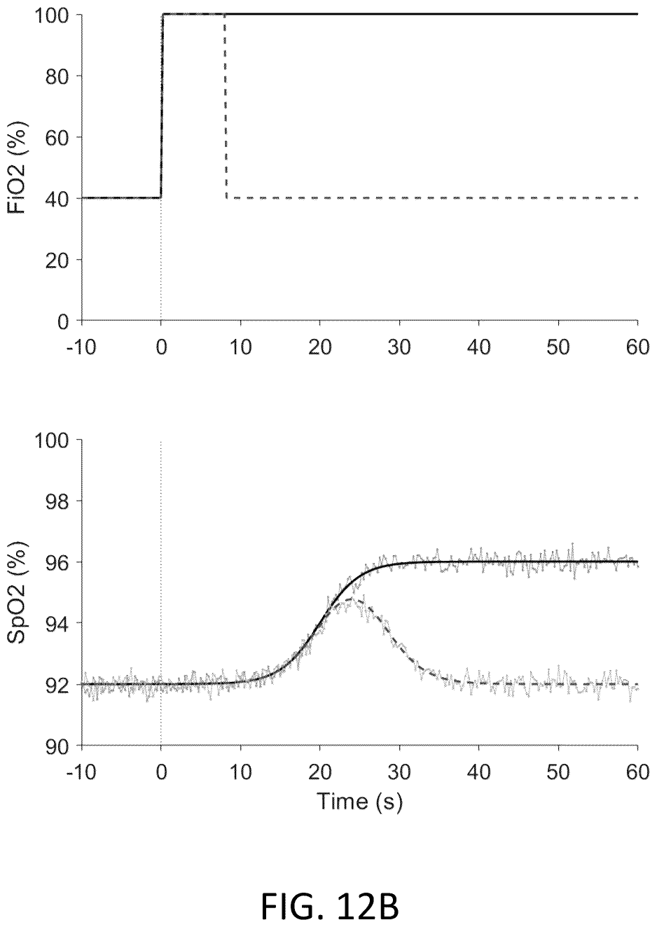

FIG. 12A illustrates another example response time procedure where the FIO₂ level is increased and then decreased in a short ‘pulse’ (e.g., less than 30 seconds or less than 10 breaths) and the patient's SpO₂ is monitored to determine the time to a peak in the SpO₂ measurement. FIG. 12B illustrates an example of using a regression-based model fit to SpO₂ measurement data from either a step response in FIO₂ or a pulse response in FIO₂.

All simulations were generated using a numerical simulation framework having a gas exchange model that receives an FIO₂ level as an input and outputs a simulated blood oxygen content provided as an arterial oxyhemoglobin saturation (SaO₂), which in turn is transformed into a peripheral oxyhemoglobin saturation value (SpO₂) using a pulse oximeter model. The gas exchange model may use a variety of factors to determine the SaO₂ value, such as any of a simulated cardiac output from the patient, hemoglobin content, physiological dead space, respiratory quotient, metabolic CO₂ production rate, body temperature, tidal volume, or respiratory rate, to name a few examples. The pulse oximeter model may also use a variety of factors to change the SaO₂ value into the SpO₂ value, such as a given physiological delay, measurement noise, or time averaging of SaO₂ values.

In FIG. 11A, the simulated SpO₂ results of three patients is provided across the execution of an example response time procedure, which occurs between the 40 minute mark and the 60 minute mark. In this example, the ventilator operates in the active period (e.g., following the physiologic closed-loop control scheme of any of FIGS. 4A-4C) from the 10 minute mark to the 40 minute mark. During this time, both the FIO₂ level on the ventilator and the simulated SpO₂ measurement from the three patients reach a steady state. For example, the FIO₂ level reaches a steady state at around 32% and the simulated SpO₂ measurement from each of the three patients reaches a steady state at around 94%. Note that both FIO₂ and SpO₂ have reached a steady state prior to the execution of the response time procedure.

Upon executing the response time procedure at the 40 minute mark, the FIO₂ physiologic closed-loop control of the ventilator is paused (although the ventilator still provides oxygen) and the response time procedure is executed. The FIO₂ level is immediately changed to a new level of 50% (although this new level could be any other value as discussed above). In this simulated example, the FIO₂ level is maintained at 50% for a first time period of 10 minutes and the SpO₂ level for each of the three patients rises to a new steady state level during this time. This time period may be a predetermined value or it may be based on the length of time it takes for the patients' SpO₂ level to reach steady state. The patient with the fastest response time (a 0 second delay in this simulation) is observed as rising the quickest to a new steady state SpO₂ level of about 96.5%, while the patient with the slowest response time (a 180 second delay in this simulation) is observed as rising the slowest to a new steady state SpO₂ level of about 96.5%. The instantaneous response time (e.g., 0 second delay) is hypothetical as there will always be at least some minimal response time delay in any real patient.

At the 50 minute mark, the FIO₂ level is changed back to its initial level of around 32% and held at this level for a second time period of 10 minutes. During this second time period, the SpO₂ level for each of the three patients falls to a new steady state level. In this example, the patient with the fastest response time is observed as falling the quickest to a new steady state SpO₂ level of about 94%, while the patient with the slowest response time is observed as falling the slowest to a new steady state SpO₂ level of about 94%. The response time procedure ends at around the 60 minute mark, and the active period is once again executed to provide closed loop control of the FIO₂ level.

FIG. 11B illustrates a closer view of the simulated results during the response time procedure between the 40 minute and 60 minute marks. A rise time, T₁ is shown for each of the three patients as is a fall time T₂. According to some embodiments, T₁ is defined as the time period between a first time when the FIO₂ level is changed to a higher value and a second time that corresponds to a percentage of the total change between the SpO₂ level at the first time and the SpO₂ level when it reaches steady state. In this example, T₁ is the amount of time between the 40 minute mark and a time when the SpO₂ level has reached 80% of its total change from 94% to 96.5% (e.g., when the SpO₂ level reaches 96%). In a similar fashion, T₂ is defined as the time period between a third time when the FIO₂ level is changed from the high value to a lower value and a fourth time that corresponds to a percentage of the total change between the SpO₂ level at the third time and the SpO₂ level when it reaches steady state. In this example, T₂ is the amount of time between the 50 minute mark and a time when the SpO₂ level has reached 80% of its total change from 96.5% to 94% (e.g., when the SpO₂ level reaches 94.5%). Note that the patient with the fastest response time has the smallest values for both T₁ and T₂ while the patient with the slowest response time has the largest values for both T₁ and T₂.

FIG. 11C provides a more detailed view of the simulated SpO₂ response of the patient with the fastest response time. The initial SpO₂ level at steady state is at 94% (SpO₂,initial), and the final SpO₂ level at steady state (SpO₂,final) is at 96.5%. The time (T₁) that it takes to reach 80% of the total change in SpO₂ (SpO₂,80%) is 80 seconds (e.g., time to reach an SpO₂ level of 96% from the moment the FIO₂ level is changed). The same process may be performed to determine the value of T₂ based on the time it takes to reach 80% of the total change in SpO₂ during its fall from 96.5% back to 94%. As noted above, any given SpO₂ response can be used for determining the values of T₁ and T₂.

In FIG. 11D, the simulated SpO₂ results of three patients is provided across the execution of a response time procedure, which occurs between the 50 minute mark and the 64 minute mark. In this example, the ventilator operates in the active period (e.g., following the physiologic closed-loop control scheme of any of FIGS. 4A-4C) from the 10 minute mark to the 50 minute mark. During this time, both the FIO₂ level on the ventilator and the simulated SpO₂ measurement from the three patients reach a steady state. For example, the FIO₂ level reaches a steady state at around 85% and the simulated SpO₂ measurement from each of the three patients reaches a steady state at the target SpO₂ of 94%. Note that both FIO₂ and SpO₂ have reached a steady state prior to the execution of the response time procedure.

Upon executing the response time procedure at the 50 minute mark, the FIO₂ physiologic close-loop control of the ventilator is paused (although the ventilator still provides oxygen) and the response time procedure is executed. The FIO₂ level is immediately changed to a new level of 70%. In this simulated example, the FIO₂ level is maintained at 70% for a first time period of 7 minutes and the SpO₂ level for each of the three patients falls to a new steady state level during this time. The patient with the fastest response time (a 0 second delay in this simulation) is observed as falling the fastest to a new steady state SpO₂ level of about 92%, while the patient with the slowest response time (a 180 second delay in this simulation) is observed as falling the slowest to a new steady state SpO₂ level of about 92%.

At the 47 minute mark, the FIO₂ level is changed back to its initial level of around 85% and held at this level for a second time period of 7 minutes. During this second time period, the SpO₂ level for each of the three patients rises to a new steady state level. In this example, the patient with the fastest response time is observed as rising the quickest to a new steady state SpO₂ level of about 94%, while the patient with the slowest response time is observed as rising the slowest to a new steady state SpO₂ level of about 94%. The response time procedure ends at around the 64 minute mark, and FIO₂ management is once again executed to using physiologic closed-loop control.

FIG. 11E illustrates a closer view of the simulated results during the response time procedure between the 50 minute and 64 minute marks. A fall time, T₂ is shown for each of the three patients as is a rise time T₁. According to some embodiments, T₂ is defined as the time period between a first time when the FIO₂ level is changed to a lower value and a second time that corresponds to a percentage of the total change between the SpO₂ level at the first time and the SpO₂ level when it reaches steady state. In this example, T₂ is the amount of time between the 50 minute mark and a time when the SpO₂ level has reached 80% of its total change from 94% to 92% (e.g., when the SpO₂ level reaches 92.4%). In a similar fashion, T₁ is defined as the time period between a third time when the FIO₂ level is changed from the low value to a higher value and a fourth time that corresponds to a percentage of the total change between the SpO₂ level at the third time and the SpO₂ level when it reaches steady state. In this example, T₁ is the amount of time between the 57 minute mark and a time when the SpO₂ level has reached 80% of its total change from 92% to 94% (e.g., when the SpO₂ level reaches 93.6%). Note that the patient with the fastest response time has the smallest values for both T₁ and T₂ while the patient with the slowest response time has the largest values for both T₁ and T₂.

In FIG. 12A, the simulated SpO₂ results of three patients is provided across the execution of another example response time procedure where the FIO₂ level is changed for a short pulse duration (e.g., for less than 30 seconds or less than 5 breaths). In this example, the ventilator may operate in the active period (e.g., following the physiologic closed-loop control scheme of any of FIGS. 4A-4C) from the 38 minute mark to about the 40 minute mark where the step change in FIO₂ occurs. During this time, both the FIO₂ level on the ventilator and the simulated SpO₂ measurement from the three patients may be in a steady state. In some examples, the ventilator operates within the response time procedure prior to the step change in FIO₂.

During the response time procedure, the FIO₂ level is immediately changed from its current level (e.g., around 31% in this example) to a new level of 50% (although this new level could be any other value as discussed above). In this simulated example, the FIO₂ level is maintained at 50% for a relatively short time period of around 20 seconds and the SpO₂ level for each of the three patients is monitored both during this time period and for some time period afterwards. For example, the patient SpO₂ may be monitored for at least 5 minutes following the end of the FIO₂ pulse.

The patient with the fastest response time (a 0 second delay in this simulation) is observed as quickly reaching a peak SpO₂ level at about 20 seconds after the FIO₂ increase, while the patient with the slowest response time (a 180 second delay in this simulation) is observed as taking longer (e.g., around 200 seconds) to reach its peak. The instantaneous response time (e.g., 0 second delay) is hypothetical as there will always be at least some minimal response time delay in any real patient. The time it takes between the initial change in FIO₂ to the peak response in patient SpO₂ is identified as T₃. In some embodiments, T₃ may be the time between the first breath taken by the patient following the change in FIO₂ and the peak response in patient SpO₂. In such an example response time procedure using an FIO₂ pulse, the time T₃ may be used as the approximation of the patient's response time. In some embodiments, rather than being a time to a peak in the SpO₂ measurement, T₃ may be the time to an initial rise in the SpO₂ measurement above a noise threshold, or a time to any other specific change in the measured SpO₂ level. It should be noted that, similar to the procedure using the longer FIO₂ step change, the change in FIO₂ may be a negative pulse (e.g., decrease followed by an increase), and the time to a negative peak in the patient's SpO₂ may be determined.

Since the SpO₂ measurement is likely to have some inherent noise in the signal, a model may be used to provide a smooth fit to the measured data. Any suitable model could be used, such as a regression-based model or least squares fit model to name some examples. FIG. 12B illustrates the use of a regression-based model to fit measured SpO₂ data during a response time procedure using a longer FIO₂ step change (solid lines) and during a response time procedure using a shorter FIO₂ pulse (dashed lines). In this example, the FIO₂ pulse lasts for around 8 seconds. The model may be used instead of the raw data when determining various characteristics of the SpO₂ data, such as when the SpO₂ reaches a steady state and/or at what SpO₂ level steady state is reached, when the SpO2 reaches a given percentage change (e.g., between its starting value and its ending steady state value), when the SpO₂ reaches a peak, or when the SpO₂ rises above a given threshold value or threshold percentage change in SpO₂, to name a few examples.

It is noted that, in some embodiments or instances, a discrepancy may exist between the FIO2 setting and the actual FIO2 of the gas leaving an air-oxygen blender of the ventilator and delivered to the patient. This may occur as a result of blender dynamics, which can cause the estimated patient response time to be less precise unless the estimation reflects this FIO2 discrepancy due to blender dynamics. In some cases, after an FIO2 setting adjustment (e.g., via closed loop control), the gas initially leaving the ventilator or air-oxygen blender will not immediately be the desired FIO2, but will over time achieve the adjusted setting, with more time potentially being required for larger adjustments. If the adjusted FIO2 setting were implemented completely and instantly, then a plot of FIO2 over time would appear as a perfect stepwise plot. In practice, the actual plotted FIO2 over time would not be a perfect stepwise plot, since there would be some time required to achieve the adjusted FIO2 setting. In some embodiments or instances, changes from an initial FIO2 setting to 100% (pure oxygen) or to 21% (room air) are performed in a stepwise or nearly perfectly stepwise manner, since these settings do not require fine-tuned adjustment of the air-oxygen blender. However, other adjustments between these values may result in delay and/or result in a dynamic approach, e.g., by a controller of the ventilator, to the desired FIO2, and may not result in a perfect or near perfect stepwise FIO2 plot. In some instances, the time required for achieving the exact FIO2 setting can be seconds or even minutes.

The simulations in FIGS. 11A-E and 12A do not reflect the above potential considerations or adjustments regarding blender dynamics, but demonstrate general concepts and system behavior. However, the FIO2 stepwise plot in FIG. 12B need not reflect these blending adjustments, since the adjustment is to an FIO2 setting of 100%.

According to some embodiments, the patient response time may be estimated with greater precision by taking into account, in some manner, the effect of blender dynamics and the delay in changing the delivered FIO2, as described above. For example, in some embodiments, estimation of patient response time may take into account signals from an oxygen concentration sensor placed at an output of the air-oxygen blender (or generally any location along the airpath between the ventilator and the patient), so as to account for any discrepancy between an FIO2 adjustment/setting and the actual FIO2 in the gas output from the air-oxygen blender following the FIO2 adjustment/setting being made.

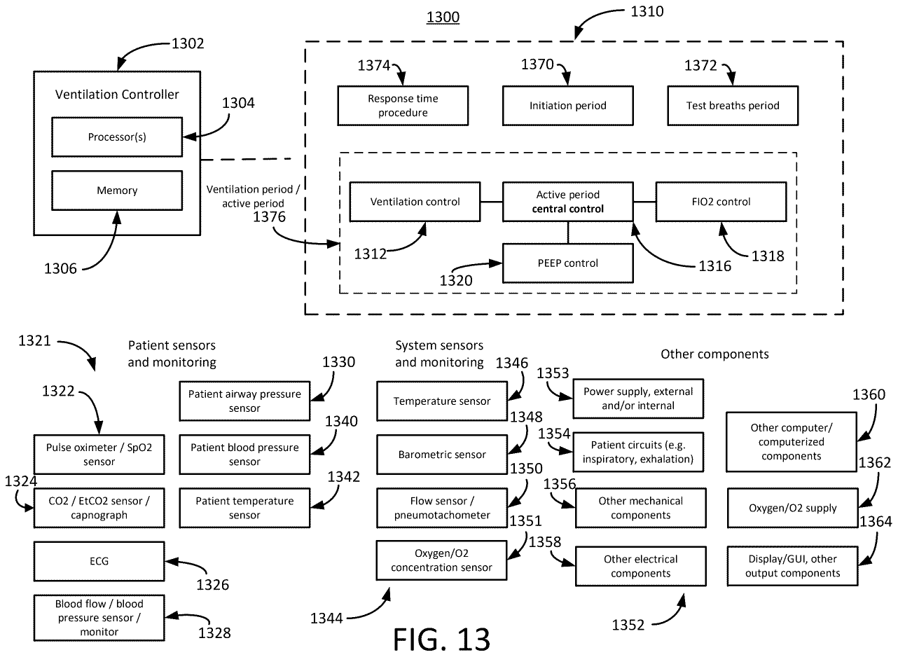

FIG. 13 illustrates an example portable ventilation system or ventilator 1300, incorporating physiologic closed-loop control, in accordance with some embodiments of the present disclosure.

The ventilation system or ventilator 1300 may include various components, such as patient sensors and monitoring components 1321, system sensors and monitoring components 1344, and other components 1351. The patient sensors and monitoring components 1321 may include an oximetry sensor 1322, such as a pulse oximeter or other sensor for providing a direct or indirect measurement, estimation or indication of oxygen saturation (SpO₂) or other blood oxygen content or concentration related parameter, a CO₂/EtCO₂ sensor/capnograph 1324, an ECG component 1326 and a patient blood flow sensor 1328. In another embodiment that ventilation and monitoring system could be the result of series of devices (ventilator, critical care monitor, other therapeutic devices and/or sensors) that work interoperably as a single life support and monitoring system.

Various combinations of these components are used in performing physiologic closed-loop control of ventilation parameters according to various embodiments, such as, for example, while ventilation is being provided to a patient via a gas delivery apparatus 106, and using a facemask 110, as depicted in FIG. 1, coupled with the patient, or using an invasive airway such as an endotracheal tube. For example, as described with reference to FIGS. 4A-4C, measured patient SpO₂ (such as may be measured, for example, using an oximetry sensor 114, as depicted in FIG. 1, such as a pulse oximeter coupled with the patient) is used in determining FIO₂ updates. In some embodiments, measured EtCO₂ (such as may be measured using a capnographic sensor 116 or capnograph, as depicted in FIG. 1, coupled with the patient) is used in determining parameter updates, including Vt, Ve, respiratory rate (RR) and PIP, such as in connection with patient hypercapnia, hypocapnia and normocapnia conditions. Patient hemodynamics, such as measures of blood flow including systolic blood pressure, may be used in connection with PEEP change eligibility (such as may be measured, for example, using a blood pressure sensor or monitor 118, as depicted in FIG. 1, coupled with the patient).

The system sensors and monitoring components 1344 may include a temperature sensor 1346, a barometric sensor 1348, flow sensor(s) 1350 such as those that utilize pneumotachometer(s), and an oxygen/O₂ concentration sensor 1351. Other components 1352 include an external and/or internal power supply 1353, patient circuits 1354, including inspiratory and exhalation circuits, other mechanical components 1356, other electrical components 1358, other computer or computerized components, such as one or more central processing units, processors and memories 1360, an oxygen/O₂ supply 1362, and a display/GUI and potentially other output components 1364.

Also included is ventilation control software 1310 with various operation modes for the ventilator including physiologic closed-loop control capability, which may be stored in whole or in part in the one or more memories 1306 of the minute ventilation controller 1302 and executed in whole or in part by the one or more processors 1304 of the physiologic closed-loop control of mechanical ventilation control system 1302.

The physiologic closed-loop control of mechanical ventilation system 1302 software includes components including an initiation period 1370, a test breaths period 1372, a response time procedure 1374, and a ventilation period/active period engine 1376. Various other software may be included, including software that may be stored or executed in whole or in part outside of the portable ventilator or ventilation system 1300.

The active period engine 1376 includes a minute ventilation control engine 1312, an FIO₂ control engine 1318 and a PEEP control engine 1320, where each or some of these engines 1312, 1318, 1320 may be in coordination with a central control engine 1316 or operate independently, and each may or may not also be in coordination with each other or some of the other engines, such as with response time procedure engine 1374. Different aspects of the physiologic closed-loop control of mechanical ventilation may be activated, for example, minute volume control 1312, FIO₂ control 1318, and/or PEEP control 1320 may be activated alone or in combination. In some embodiments, FIO₂ closed-loop control is activated without PEEP closed-loop control, such that PEEP is manually adjusted by the user; or the minute volume closed-loop while the user has responsibility for the control of FIO₂ and PEEP.

As discussed above, FIO₂ physiologic closed-loop control can provide advantages including minimizing the time that a patient's SpO₂ is below or substantially below a target value, providing rapid oxygenation response to patient SpO₂ desaturation events and helping prevent patient hypoxia/hypoxemia and hyperoxia/hyperoxemia. Furthermore, incorporating the patient response time into the FIO₂ closed-loop control allows for some patients (e.g., those with faster response times) to be brought to their target SpO₂ level even faster. The patient response time may be determined by ventilator 1300 by way of response time procedure engine 1374.

It is noted that, while the modules and engines are depicted separately for conceptual purposes, they may be implemented in a combined, integrated or different manner, such as embodiments in which aspects of the roles of each of the operational phases or engines are distributed differently or combined in various ways. Moreover, other conceptual frameworks may also be utilized in various embodiments.

In some embodiments, the ventilator system or ventilator 1300 may be operated in one of several control schemes based on the user, patient and/or area or circumstance of operation (e.g., remote environment rescue, aeromedical transport, prolonged field care, etc.). One (or several) such schemes may include physiologic closed-loop control of ventilation, PEEP, and FIO₂, which may collectively be referred to as a VPFC system. According to some embodiments, initiation period 1370, test breaths period 1372, active period (or ventilation period) 1376, and response time procedure 1374 may occur in a particular order and represent phases of operation in the VPFC system. In some embodiments, after turning on the ventilator 1300, the user may select the VPFC system from amongst other types of control schemes. However, embodiments are contemplated in which VPFC system is entered into or begun without user selection or input.

In various embodiments, various types of physiologic closed-loop control, such as the VPFC system, can be started or engaged, as well as stopped or disengaged, in different ways. In some embodiments, VPFC or other aspects of physiologic closed-loop control may start automatically, such as at the onset of active period ventilation. In some embodiments, the ventilator 1300 may include a control component, such as a physical button, control knob or graphical user interface (GUI) component, which the user may press, engage or actuate in order to start, select or turn on the VPFC system or a particular aspect or engine of physiologic closed-loop control based on the patient's condition, or the circumstances referenced above. For example, in some embodiments, the user may be provided with options to select and initiate, for example, specific VPFC system engines, closed-loop control of FIO₂ but manual PEEP control or adjustment, or closed-loop control of minute ventilation and FIO₂ but manual PEEP control or adjustment. Furthermore, in some embodiments, the user may be provided with options to start, stop or re-start various types of closed-loop control at different times, so that the VPFC system or other aspects of closed-loop control are available, but also subject to disengagement, and so are made to be essentially on-demand as required to manage the patient or circumstance. Also, as mentioned previously, in some embodiments, during operation of aspects of closed-loop control, the user may be able to change a particular setting or settings, such as FIO₂ or PEEP, and then the physiologic closed-loop control may resume from that point and initially with that setting or those settings as could be the case when the patient requires additional FIO₂ to facilitated endotracheal suctioning. Furthermore, in some embodiments, a user may be provided with an option to stop or disengage the VPFC system or some other aspect of physiologic closed-loop control (or the physiologic closed-loop control may be automatically stopped), and later the user may restart it as could be the case when the patient is disturbed during movement to or from a vehicle which could cause transient physiologic disturbances. These features could also trigger automatically when an accelerometer(s) detect characteristic movement patterns. In some embodiments, a user may select to initiate the response time procedure 1374, thus pausing any physiologic closed-loop control being performed within active period 1376 and returning to the physiologic closed-loop control upon the completion of the response time procedure 1374. Still further, in some embodiments, multiple or different users may oversee or operate the ventilator, such as at different times, and each user may take different actions.

In some embodiments, for example, a user, such as a minimally trained user, may initiate active period ventilation with the VPFC system (or another aspect of physiologic closed-loop control). At some point during active period ventilation, that user or another user, such as a more trained user, may change from the full VPFC system (or another aspect of physiologic closed-loop control) to manual, or more manual, operation. Alternatively, active period ventilation may be initiated without the VPFC system or some other aspect of physiologic closed-loop control, and the VPFC system or some other aspect of physiologic closed-loop control may be started or engaged later, such as if the user becomes distracted (or during the period of user distraction), or by a less trained user who may later arrive at the scene, for example. In some embodiments, the VPFC system may activate automatically if the sensor/monitoring system detects a physiologic pattern that represents a threat to the patient's wellbeing. Such a case could be the automated initiation of FIO₂ physiologic closed-loop control when the ventilator system detects significant changes is altitude which would be associated with aeromedical transport. In this case, the VPFC system would initiate the FIO₂ control engine to ensure that the patient's oxygen saturation is maintained as the cabin pressure is reduced during the flight.

The following provides an exemplary overview of operation of the VPFC system, according to some embodiments. Further details, regarding various aspects and in various embodiments, are provided with reference to later figures.

In some embodiments, at the start of the execution of initiation period 1370, or after the user selects the VPFC system, the user may be prompted (via a display of the ventilator 1300) to enter the patient's sex and height. From this, the controller 1302 may determine an associated bodyweight, which may be called the patient's PBW. However, the determined bodyweight may not be the patient's actual bodyweight, and may represent an ideal bodyweight, approximated, typical or other associated bodyweight to be used, for example, in determination or optimization of certain ventilation settings. Predicted bodyweight may be used, as opposed to actual bodyweight, for example, since actual bodyweight may fluctuate substantially from person to person, and even between individuals of similar characteristics such as height and sex, due to different amounts of muscle and fat from person to person, while lung volume, capacity and function remain relatively unaffected by such factors. As such, in some embodiments, PBW may be used at least in part as an indicator associated with an individual's respiratory capacity, and may generally be a better indicator than actual patient bodyweight.

During the execution of initiation period 1370, the controller 1302 may utilize the PBW in determining one or more ventilation parameter settings to be used for one or several operational breaths to be delivered to the patient, which may be utilized, for example, in confirming correct operation of the ventilator 1300, which may include confirming that there are no leaks, or significant leaks, in the circuit. For example, PBW may be used in setting initial Vt, such as may be set as Vt (in ml)=A*PBW (in kg), where A may be, e.g., 5, 6, 7, 8 or 9 (in ml/kg). Furthermore, for example, initial minute volume (V_{M} in ml/min) may be set as B*PBW, where B (in ml/(min*kg) may be, e.g., 80, 90, 100, 110, 120 or 150.

Once the execution of the initiation period 1370 is successfully completed, the ventilator 1300 may execute test breaths period 1372. During the execution of test breaths period 1372, one or more test breaths are delivered to the patient and respiratory data is obtained. The respiratory data is used in determining one or more patient respiratory parameters, such as estimated patient respiratory system compliance (Crs) and, in some embodiments, one or more additional parameters, such as estimated patient respiratory system resistance (Rrs). In some embodiments, if Crs cannot be estimated or sufficiently estimated then a default value, such as 100 ml/cm H₂O may be used. The determined patient Crs is then used in determining an initial peak inspiratory pressure (PIP(0)) setting to be used at the start of active period 1376.

In some embodiments, the ventilator 1300 may execute response time procedure 1374 following the completion of test breaths period 1372 and before the execution of the active period 1376. The patient response time may be determined by response time procedure 1374 as discussed above with reference to FIGS. 9 and 10. In some embodiments, the ventilator 1300 first proceeds to active period 1376 following the completion of test breaths period 1372 and proceeds from active period 1376 where the response time procedure 1374 activates once the FIO₂ and SpO₂ levels have reached a steady state during the physiologic closed-loop control of active period 1376. Ventilator 1300 may pause the physiologic closed-loop control during active period 1376 at any time to execute response time procedure 1374, returning to active control 1376 at the conclusion of the response time procedure 1374.

In some embodiments, active period 1376 may include physiologic closed-loop control of FIO₂ and PEEP, and possibly one or more other parameters. During physiologic closed-loop control of mechanical ventilation 1376, active period control 1316, in coordination with minute volume control 1312, FIO₂ control 1318 and PEEP control 1320, sets or adjusts ventilation settings including FIO₂, PEEP, PIP, Vt, V_{M}, RR, and/or inspiratory:expiratory ratio (I:E). However, in some embodiments, one or more of minute volume control 1312, FIO₂ control 1318, PEEP control 1320, or other components, may set or adjust, or participate in the setting or adjusting of, certain of those or other ventilation settings.

As described above, the controller 1302 may utilize a target SpO₂, such as 94% (however, in various embodiments, the target SpO₂ may be, e.g., between 92%-98%, between 92%-96% or between 96%-98%). On a continuous basis, actual measured patient SpO₂ may be used as input in adjusting the FIO₂ setting of the ventilator as described above with reference to FIGS. 4A-4C.

In some embodiments, determined parameters including patient end-tidal carbon dioxide (EtCO2), a determined patient airway pressure waveform, patient airway flow waveform (\dot{V}), and volume waveform (V) may be used in determining active period initial settings for Vt, Ve, PIP, RR and I:E, and/or, in some embodiments, parameters can be used that are derived at least in part from the \dot{V} and V waveforms, which can include plateau pressure, driving pressure, Crs and Rrs. Furthermore, the controller 1302 may continuously use determined patient Crs and Rrs in determining adjustments to Vt, such as may maintain Vt at a safe level. The controller 1302 may also continuously adjust RR, such as to maintain V_{M} at a constant level when Vt is adjusted, where Ve=Vt*RR.

FIG. 14 is a flow diagram illustrating aspects of a method 1400 of operation of an example initiation period of a physiologic closed-loop mechanical ventilation system incorporating a series of closed-loop control engines, in accordance with some embodiments of the present disclosure. The purpose of this initiation period may be to ensure that the ventilator is able to operate under normal parameters and limits specifically related to the patient and the physiologic manifestations of their underlying disease state.

At operation 1402, a user selects a to initiate the physiologic closed-loop control (PCLC) of mechanical ventilation system. However, in some embodiments, no such user selection is required. At operation 1404, operational breaths are administered, such as, for example, via a gas delivery apparatus 106 and using a facemask 110, as depicted in FIG. 1, coupled with the patient, or using an invasive airway such as an endotracheal tube, using initial values for parameters including Vt, RR, I:E, PEEP and FIO₂.

At operation 1406, the method 1400 queries whether there is a PEEP leak, such as, for example, by determining whether measured proximal airway pressure is steady at the end of expiration. This may be done, for example, by determining that airway pressure has not decreased by more than a particular threshold amount or percentage at the end of an expiration. If measured proximal airway pressure is not steady at the end of expiration, then, at operation 1408, an alarm, such as a PEEP leak alarm, is triggered, and the method 1400 returns to operation 1404. If measured proximal airway pressure is held steady at the end of expiration as a result of a user action to correct the leak or the user's activation of leak compensation, then, at operation 1410, the method 1400 queries whether PIP is under limit, such as under 35 cm H₂O (or, e.g., between 30-40 cm H₂O), for a certain number of consecutive operational breaths, such as 2 (however, in various embodiments, the number of breaths may be, e.g., 1-10). If not, then, at operation 1412, a high PIP alarm is triggered. If so, then initiation period is complete and, at operation 1414, the method 1400 proceeds to test breaths period of operation.

FIG. 15 is a flow diagram illustrating aspects of a method 1500 of operation of an example test breaths period of a PCLC mechanical ventilation system incorporating closed-loop control, in accordance with some embodiments of the present disclosure. In some embodiments, during execution of the test breaths period, the system may deliver a series of breaths, and based on associated collected data, determine an initial PIP(0) setting to be used in active period, as well as ensure that the system is leak-free or that active compensation is maintaining the baseline airway pressure.

At operation 1502, breaths are administered, such as, for example, via a gas delivery apparatus 106 and using a facemask 110, as depicted in FIG. 1, coupled with the patient, or using an invasive airway such as an endotracheal tube, using initial values for RR, Ti, I:E, PEEP and Vt. In some embodiments, the target Vt may be determined based on the patient's predicted body weight, as input from the patient's height and gender. At operation 1504, the method 1500 queries whether any leaks are detected, such as, for example, by determining if the measured airway pressure is decreasing at the end of expiration or by determining whether a detected amount of flow is not equal to, or not close enough to, the amount that is set to be initially provided by the ventilator. If so, then, at operation 1506 a leak alarm is triggered. In some embodiments, if the leak alarm is triggered, the system may continue delivering ventilation with current settings until further action is taken. If not, then, at operation 1508, the method 1500 queries whether the delivered Vt is less than the target Vt.

If Vt is not less than the target Vt, then, at operation 1510, PIP(0) is set based on one or more last breaths. For example, in some embodiments, PIP(0) is calculated by interpolation between the current and previous PIP settings, but, in various embodiments, different models or calculation methods may be used. At operation 1518, test breaths period is complete and either active period or response time procedure can be entered.

If Vt is less than the target Vt, then, at operation 1512, the method 1500 queries whether PIP is below threshold. If so, then, at operation 1514, PIP is increased, and the method 1500 proceeds back to operation 1502. If not, then, at operation 1516, PIP(0) is set to a particular value. At operation 1518, test breaths period is complete and active period or response time procedure can initiate.

In some embodiments, the first breath will have a PIP of 10 cm H₂O (however, in various embodiments, the first breath may have a PIP of, e.g., 5-15 cm H₂O), which may increase by 3 cm H₂O for each subsequent breath up to a maximum of 30 cm H₂O (however, in various embodiments, the maximum may be, e.g., 25-35 cm H₂O) or the maximum PIP minus 5 cm H₂O. Once the delivered volume is greater than or equal to the target volume, the breaths are stopped and initial PIP is calculated by interpolation between the current and previous PIPs. If a PIP of 30 cm H₂O does not achieve the desired target volume, the initial PIP(0) is set to 30 cm H₂O (however, in various embodiments, the PIP(0) may be set to, e.g., 25-35 cm H₂O).

FIG. 16 is a flow diagram illustrating aspects of an example desaturation related method 1600, in accordance with some embodiments of the present disclosure, which may, for example, be implemented by a ventilation system as disclosed herein.

At operation 1602, the system confirms that the patient SpO₂ signal is valid. If not, FIO₂ PCLC (e.g., active FIO₂ control engine) is paused, such as until a valid signal is obtained. If the SpO₂ signal is validated, then the method 1600 proceeds to operation 1604, where the system confirms that the ventilation system is confirmed as operational. Here also, if the ventilation system cannot be confirmed as operational, then, at operation 1618, FIO₂ PCLC is paused, such as until the validation can be obtained. If the ventilation system is confirmed as operational, then the method 1600 proceeds to operation 1606.

At operation 1606, the method 1600 queries whether SpO₂ (such as may be measured, for example, using an oximetry sensor 114, as depicted in FIG. 1, such as a pulse oximeter coupled with the patient), is below a threshold (e.g., 88%) for at least a threshold period of time, such as 0.25 seconds (or, e.g., between 0.25-1 second, between 1-5 seconds, between 5-15 seconds or between 15-30 seconds). If not, then, at operation 1620 FIO₂ PCLC (e.g., aspects of one implementation of which are described with reference to any of FIGS. 4A-4C) is enabled (or continued) and, at operation 1622, the method 1600 waits another increment of time. In some embodiments, the threshold period of time may function as a check to ensure that a determined very low SpO₂ is not incorrectly representative of the patient's SpO₂, such as by being caused by movement, incorrect probe placement or artifact.

If, at operation 1606, it is determined that SpO₂ is below the threshold level for at least the threshold period of time, then, at operation 1608 FIO₂ is set to 100% (however, in various embodiments, the new FIO₂ setting may be, e.g., 50%-100%), and the process proceeds from there. At operation 1610, an alarm is displayed to inform the user that the desaturation threshold has been reached. In some embodiments, this may be done in order to urgently react to and minimize the duration and/or severity of the desaturation event.

Next, at operation 1612, the method 1600 queries whether more than one desaturation event (however, in various embodiments, the number of desaturation events may be greater than 1, such as 2-5) has occurred in a threshold period of time (e.g., the last 20, 40 or 60 minutes). If not, then, at operation 1622, the method 1600 waits another increment of time.

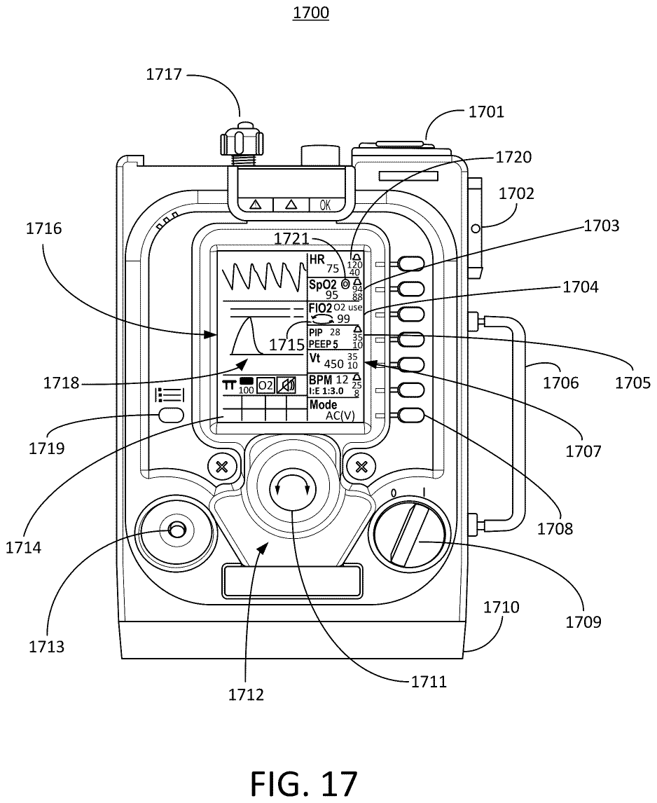

At operation 1612, if it is determined that desaturation has occurred more than once in a threshold period of time (e.g., the last 20, 40 or 60 minutes), then, at operation 1614, the target SpO₂ is increased by a set amount, such as +1% (however, in various embodiments, the amount may be, e.g., between +1% and +3%) and, at operation 1616, an alarm is displayed to alert the user of this development (such as via a GUI on a display of a portable ventilator 1700 as described herein with reference to FIG. 17). Next, at operation 1622 the method 1600 waits another time increment. In another embodiment, following a threshold period of time, the target SpO2 may be decreased by 1% based on the operational need to drastically conserve oxygen while maintaining normoxia/normoxemia. In these environments the target SpO₂ could be lowered to a range between 90-92%.

FIG. 17 illustrates an example portable ventilator 1700, in accordance with some embodiments of the present disclosure. Portable ventilator 1700 includes features such as pulse oximeter connector 1701, fresh gas/emergency air intake 1702, handle 1706, power switch 1709, battery compartment 1710, user selection dial 1711, control panel 1712, manual breath/plateau pressure button 1713, menu button 1719, parameter buttons 1708, oxygen inlet 1717 and a display and user interface screen 1716.

The fresh gas/emergency air intake 1702 provides a gas path and allows ambient air into the device's internal compressor. Built-in filters are used to protect the compressor and patient from particulate matter. The intake 1702 also acts as an anti-asphyxia path that enables the patient to breathe ambient air, should the ventilator fail. The intake 1702 further contains a particulate filter and permits the user to connect either a bacteria/viral or a chemical/biological filter, depending on ambient conditions. Furthermore, an oxygen reservoir bag assembly may be connected to the intake 1702 to allow for low-flow oxygen use with the ventilator 1700 in order to provide a source of supplemental oxygen to patients during ventilation. For example, low-flow oxygen sources can be obtained based on a flow meter or an oxygen concentrator. Oxygen may be delivered through the intake 1702 when the ventilator's internal compressor cycles deliver breaths.

A top panel of the ventilator 1700 may have components including, in addition to the intake 1702 and the pulse oximeter connector 1701, a high-pressure oxygen input, a gas output, a power cord connector for external AC/DC power, a USB port, an exhalation valve port, an exhaust valve and a pressure transducer port. The pulse oximeter connector 1701 is used to connect a pulse oximeter that may provide continuous non-invasive monitoring of SpO₂ and pulse rate. The portable ventilator 1700 may be operable using external AC/DC power or a battery, such as an internal lithium ion battery.

Furthermore, the portable ventilator 1700 may include at least one display and user interface screen 1716, which may, for example, include a liquid crystal display (LCD). Among other things, the display and user interface 1716 may provide a user with data relating to patient parameters and ventilation parameters, including current ventilator settings, which may be continuously updated. Furthermore, the display and user interface 1716 may include, among other things, various GUI aspects, allowing user interaction, such as to access particular data, change ventilator selections or settings, confirm suggested displayed changes to ventilator settings, receive and respond to alarms, etc. In particular, as shown, the display and user interface screen 1716 includes parameter and alarm indicators 1707, an alarm message center/waveform window 1718, and auxiliary parameter boxes 1714.

In some embodiments, the display and user interface screen 1716 may be divided into a number of sections. For example, as depicted, the top left area of the display and user interface screen 1716 may include airway pressure, flow, volume, capnography and plethysmography waveform plots. This section may include displayed plots for airway pressure as well as, when a pulse oximeter is connected, the plethysmography waveform, and when a CO₂ sensor is connected, the capnogram. When a plot is useful to facilitate a parameter adjustment by the user, a message area may display both the plot and a context menu that the user may use to make selections to obtain displayed context relating to the parameter.

The display and user interface screen 1716 also includes a menu display section in the top left area. This section may be used to display a menu after the user presses a menu button on the ventilator's control panel, and may be used to display context menus associated with the operational status of the ventilator, alarm configuration, alarm history, audible alarm volume, LCD brightness/contrast, etc.

The display and user interface screen 1716 also includes an alarm message center/waveform window 1718 in the upper left area, in which visible alarms may at times be displayed. Some alarms may instruct the user to consult a physician, for example. In some embodiments, alarms may be categorized into different levels of priority, such as based on the level and/or urgency of the risk that the particular alarm condition may pose to the patient. Multiple alarms, along with their priorities, that have occurred recently may be available for display to a user, where the user may view the recent alarms by scrolling in a GUI, for example. In some embodiments, if the FIO₂ setting is increased by a certain amount, such as 10% (or, e.g., 5-15%) during a predetermined period of time, such as 10 minutes (or, e.g., 5-15 minutes), an alarm is generated. Furthermore, in some embodiments, certain alarms may cause some or all physiologic closed-loop control aspects, or VPFC system including FIO₂, minute volume, and PEEP closed-loop control engines, to pause until the user clears the alarm. In some embodiments, during the time of the pause and before the alarm is cleared, the ventilator may operate using current parameter values, such as for FIO₂ and PEEP, that were being used at the time that the pause was initiated.

The display and user interface screen 1716 may also include pop-up windows that may provide a user with context-sensitive guidance, such as in connection with manual adjustment of parameter values, for example.

The display and user interface screen 1716 also includes various parameter windows on the right side. Displayed parameters may include, e.g., SpO₂, EtCO2, FIO₂, PEEP, PIP, Vt, BPM and blood pressure, for example. In some embodiments, the patient's measured response time may be displayed in one or more of the parameter windows. Each parameter window may display a primary parameter as well as secondary parameters, such as parameters that may be related to the primary parameter or with associated alarm limits. In some embodiments, solid text may be displayed for primary and secondary parameter values that can be adjusted by the user, while outlined text may be used for patient-dependent measured parameters, for example. Primary parameters may also include mode, which may include a user selectable mode of operation including assist/control (AC), synchronized intermittent mandatory ventilation (SIMV), continuous positive airway pressure (CPAP) and bilevel (BL).

Furthermore, the mode parameter may be associated with secondary parameter choices including volume targeting and pressure targeting. Volume targeting aims to deliver a constant volume to the patient in the inspiratory time using a constant flow. During volume targeting, the measured PIP parameter is displayed or highlighted. Pressure targeting aims to achieve a constant airway pressure for the duration of the inspiratory time. During pressure targeting, the measured Vt parameter is displayed or highlighted.

The display and user interface screen 1716 also includes device-related icons section in the lower left area. This section may include icons that represent, and may provide status information on, for example, the ventilator's power source (which may indicate whether the ventilator is operating on external power or its battery), a battery charging status icon, an oxygen supply attachment icon, and an icon that indicates whether audible alarms or permitted or muted.

The display and user interface 1716 may also include an auxiliary parameter boxes section 1714, which may be located toward the bottom. This section may display parameter boxes that allow the user to adjust a particular parameter using a context menu associated with the parameter.

In some embodiments, a user may take the following steps in setting up. The patient circuit may be attached to the ventilator's top panel. A high-pressure oxygen supply, if it is to be used, is attached. The user inspects the fresh gas/emergency air intake filters and may attach other items, such as an oxygen reservoir bag, and biological and chemical filters. The user may choose a power source, such as an external or internal power source. The user may connect the power supply to the ventilator. Once preliminary steps are completed, the user may power on the ventilator 1700 using the ventilator's power switch or button. Once powered on, the ventilator 1700 may perform a self-check, to check for potential alarm conditions as well as the operation of the pneumatic system, power system and internal communications system. During normal start-up, the ventilator's alarms may be muted for, e.g., 2 minutes, to allow the user to connect items including the patient circuit and pulse oximeter, and to perform operational tests.

In some embodiments, after powering on the ventilator 1700, the user may choose from the settings defaults, such as adult, pediatric, mask CPAP, custom (includes use of saved settings values), and last settings (includes use of last-used settings values). Alternatively, these setting defaults would include VPFC or a subset of the physiologic closed-loop control engines described herein. The user may select one of the defaults, in which case ventilation will be initiated using the default settings associated with the selection. Alternatively, the user may manually set settings using any of the parameter buttons 1708. Furthermore, the user may select a mode of operation, including, as described briefly above, AC, SIMV, CPAP or bilevel. In AC, the patient receives either controlled or assisted breaths. When the patient triggers an assisted breath, the patient receives a breath, and either a pressure target or a volume target is utilized. In SIMV, the patient receives controlled breaths based on the setting of the breathing rate. In CPAP, the patient receives a constant positive airway pressure while breathing spontaneously. Spontaneous breaths may be either unsupported demand flow or supported using pressure support. In bilevel mode, the ventilator 1700 provides two pressure settings to assist the patient in breathing spontaneously, including a higher inspired positive airway pressure (IPAP) and a lower expiratory positive airway pressure (EPAP).

The parameter and alarm indicators 1707 may include information specifying current parameter values, and may also display other related information such as alarm threshold values 1720, which may indicate, for example, threshold beyond which an alarm may be triggered. The parameter and alarm indicators 1707 may include an SpO₂ parameter display aspect 1703, an FIO₂ parameter display aspect 1704 and a PEEP parameter display aspect 1705.

In the SpO₂ parameter display aspect 1703, the current patient SpO₂ is displayed as “95”, meaning 95%. A target symbol 1721, along with the displayed numbers “94” and “88” indicate that the SpO₂ target is set to 94% and the desaturation threshold of SpO₂ is set to 88%.

In the FIO₂ parameter display aspect 1704, the current FIO₂ is displayed as “99”, meaning 99%. Double arrows 1715 (which, in some embodiments, may be animated as displayed), indicate that FIO₂ PCLC is currently turned active and operating. The displayed “O₂ Use” text indicates that an attached oxygen supply is being used and the rate of oxygen use in L/min.

In some embodiments, various alerts or warnings may be displayed to the user on the display and user interface screen 1716 before the user initiates FIO₂ PCLC. For example, the user may be warned not to use FIO₂ PCLC if the user suspects that pulse oximetry may not operate correctly or may not be available, or if the patient has carboxyhemoglobin poisoning (i.e., carbon monoxide poisoning), in which case the user may be advised to follow the local standard of care. The user may also be warned not to use FIO₂ PCLC for patients with a core temperature of less than 35 degrees Celsius. Furthermore, in some embodiments, pulse oximetry and a high-pressure oxygen source may be required for FIO₂ PCLC, and failure of availability of either of these resources may cause FIO₂ PCLC to be paused and may cause an associated alarm to be displayed to the user.

In the PIP parameter display aspect 1705, the current PIP is displayed as 28, meaning 28 cm H₂O, and the current PEEP is displayed at 5, meaning 5 cm H₂O. PIP alarm threshold levels are also displayed.

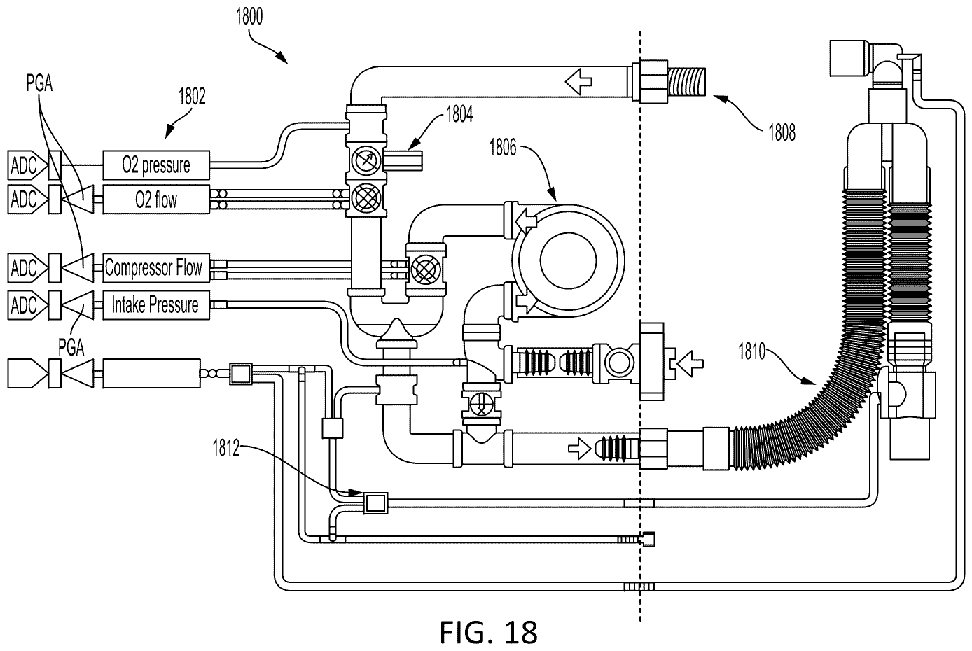

FIG. 18 illustrates aspects of an example pneumatic system 1800 that can be used with a portable ventilator, in accordance with some embodiments of the present disclosure. As shown, the pneumatic system 1800 includes a high-pressure oxygen connector 1808, patient circuit tubing 1810 that may include patent inhalation and exhalation circuit components, an exhalation valve 1812, a radial compressor 1806 or other gas moving component such as a blower, an oxygen valve 1804 and components 1802 associated with control and/or measurement of oxygen pressure, oxygen flow, ambient pressure, air flow, intake pressure and patient airway pressure, which may include programmable-gain amplifiers (PGAs), analog-to-digital converters (ADCs), and other components.

The oxygen valve 1804 and the compressor 1806 may provide the appropriate gas mixture for the patient during ventilation. The pneumatic system 1800 may include transducers for pressure measurements including oxygen input supply and barometric pressure. The patient circuit tubing 1810 may include an inspiratory portion that provides gas to the patient, as well as an expiratory portion that exhausts gas directly to the atmosphere without return to the pneumatic system 1800. The pneumatic system 1800 pneumatically controls the exhalation valve 1812. A transducer within the pneumatic system 1800 measures the airway pressure during ventilation.

An external high-pressure oxygen source connects to the pneumatic system 1800 using a high-pressure oxygen input port. The source may be a medical grade piped oxygen system or oxygen cylinder supply, for example.

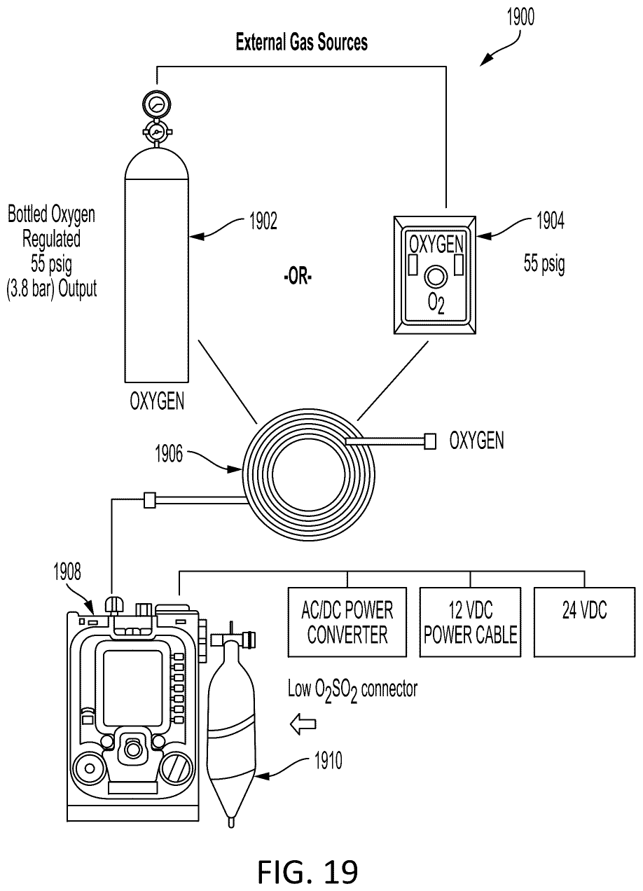

In some embodiments, a portable ventilator, such as, for example, the portable ventilator 102, as depicted in FIG. 1, or the portable ventilator 1700, as depicted in FIG. 17, may be coupled with a supplemental oxygen source (some examples of types of oxygen sources are provided with reference to FIG. 19). In various embodiments, the portable ventilator may be capable of supplying oxygen, using the supplemental oxygen source, in a number of different ways, or the portable ventilator may be capable of supplying oxygen in any one of several different ways. In various embodiments, the manner in which oxygen is supplied may be determined without user selection, or may be selected by a user, and may or may not require user confirmation.

In some embodiments, a reservoir bag may be used that allows entrainment of oxygen from a low-pressure oxygen source. For example, a user may adjust the flow rate of oxygen based at least in part on current SpO₂ (such as, for example, may be measured using an oximetry sensor 114, as depicted in FIG. 1, such as a pulse oximeter coupled with the patient).

In some embodiments, the portable ventilator includes and uses a variable rate regulatory valve, in which an oxygen output rate allowed or facilitated by the variable rate regulatory valve may be varied and changed to a particular oxygen flow rate of a range of possible oxygen flow rates, such as may range from 0 l/min to 200 l/min (or, e.g., up to 220, 250 or 275 l/min). The variable rate regulatory valve may be used in providing a variable and controllable oxygen flow rate for gas provided by a gas delivery apparatus (such as, for example, gas delivery apparatus 106, as depicted in FIG. 1) of the portable ventilator, such as using a facemask 110, as depicted in FIG. 1, coupled with the patient, or using an invasive airway such as an endotracheal tube, during the providing of mechanical ventilation. In some embodiments, the variable rate regulatory valve may attach to a gas inlet (such as, for example, oxygen inlet 1717, as depicted in FIG. 17) of the portable ventilator and a high-pressure oxygen source, and potentially one or more other devices and/or sensing or monitoring components.

In some embodiments, the variable rate regulatory valve may be controlled automatically or without need for user interaction. For example, this may be based at least in part on the patient's continuously monitored SpO₂ and in accordance with an FIO₂ setting or adjustment that may be determined based at least in part on the current SpO₂. In some embodiments, this arrangement may be used in providing FIO₂ CLC. In some embodiments, a portable oxygen concentrator (POC) may be used. FIO₂ CLC may be used in controlling and regulating the output of the POC for entrainment of oxygen into the gas delivery apparatus during mechanical ventilation. Furthermore, in some embodiments, including embodiments in which the variable rate regulatory valve or a POC is used, PEEP CLC may also be included in control of the gas delivery apparatus. In some embodiments, PEEP CLC may be based least in part on a current FIO₂ setting and a current PEEP setting.

FIG. 19 illustrates aspects of an example external gas supply system 1900 that can be used with a portable ventilator 1908, in accordance with some embodiments of the present disclosure. Depicted components include an oxygen tank 1902 or other oxygen output 1904, high pressure hose 1906 for use with the oxygen supply, and low flow oxygen source 1910 shown coupled to the portable ventilator 1908.

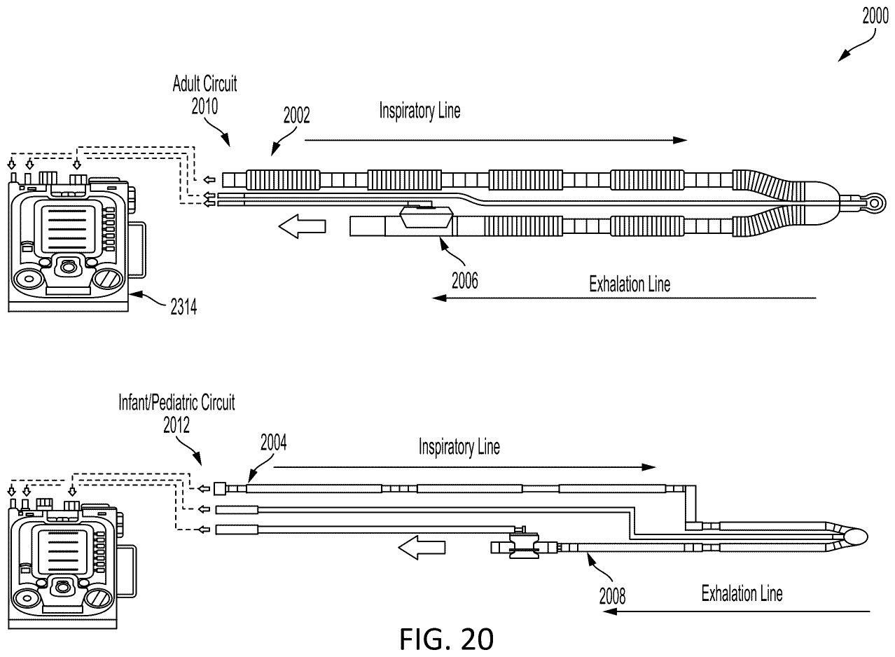

FIG. 20 illustrates aspects of example patient circuits 2000 that can be used with a portable ventilator 2014, in accordance with some embodiments of the present disclosure. Depicted components include an adult circuit 2010, including an inspiratory line 2002 and an expiratory line 2006, and an infant/pediatric circuit 2012, including an inspiratory line 2004 and an expiratory line 2008.

Various embodiments described herein may apply to, for example, out-of-hospital or pre-hospital patient care (although not limited to such care). Some aspects of embodiments described herein take into account practical factors relating to such care contexts. For example, in pre-hospital patient care, the care provider may be less trained than a hospital-based care provider. Furthermore, no support or less support from other care providers and/or hospital systems may be available. Also, pre-hospital care may involve less patient and equipment physical stability, and may involve movement or transport. Additionally, pre-hospital patient care may extend for long periods of time, sometimes extending to many hours, a day or several days. During this time, the resulting physical and mental stress, and overall exhaustion, of the patient and the care provider can be significant factors to consider in determining optimal ventilator operation, procedures and processes. As such, in some embodiments, these pre-hospital conditions are taken into account, such as in connection with determination of parameter values, ranges and thresholds, frequency of change of parameters, overall simplicity, and balances between parameter stability and the need for adjustment. In some embodiments, such balances and optimization approaches may be thought of as providing or favoring a form of conceptual “guardrails” in view of the particular concerns and elevated potential risks that tend to accompany pre-hospital care contexts and situations.

1. A mechanical ventilator apparatus for customizing therapeutic delivery of gas to a patient, comprising:
a gas delivery apparatus, having a patient interface, configured to deliver the gas to the patient;
an oximetry sensor configured to generate signals representative of an oxygen concentration of the patient's blood; and
a controller, comprising a processor and a memory, in communication with the gas delivery apparatus and the oximetry sensor, the controller configured to:
receive the signals representative of the oxygen concentration of the patient's blood,
determine the oxygen concentration of the patient's blood based at least in part on the received signals,
provide a test change in an FIO₂ setting of the gas being delivered to the patient,
determine a patient response time based at least in part on the oxygen concentration of the patient's blood in response to the test change in the FIO₂ setting, and
adjust the FIO₂ setting based at least in part on the determined patient response time and the oxygen concentration of the patient's blood.

2. The mechanical ventilator apparatus of claim 1, wherein the patient response time is specific to the patient.

3. The mechanical ventilator apparatus of claim 1, wherein the controller is configured to determine an FIO₂ correction value and to adjust the FIO₂ setting based at least in part on the FIO₂ correction value.

4. The mechanical ventilator apparatus of claim 3, wherein the FIO₂ correction value is inversely proportional to the patient response time.

5. The mechanical ventilator apparatus of claim 3, wherein the FIO₂ correction value includes a proportional gain term and a derivative gain term.

6. The mechanical ventilator apparatus of claim 5, wherein the controller is configured to determine the derivative gain term by determining a rate at which an SpO₂ level representative of the oxygen concentration of the patient's blood changes over a set period of time.

7. The mechanical ventilator apparatus of claim 5, wherein the controller is configured to determine the proportional gain term by determining both a response time coefficient representative of the patient response time and an error term representative of a difference between an SpO₂ level representative of the oxygen concentration of the patient's blood and a target SpO₂ level.

8. The mechanical ventilator apparatus of claim 7, wherein the controller is configured to apply a first proportional gain coefficient to the response time coefficient and error term if the SpO₂ level is within a predetermined range, and is configured to apply a second proportional gain coefficient to the response time coefficient and error term if the SpO₂ level is outside of the predetermined range.

9. The mechanical ventilator apparatus of claim 8, wherein the predetermined range is between 93% and 99%.

10. The mechanical ventilator apparatus of claim 8, wherein the second proportional gain coefficient is twice the magnitude of the first proportional gain coefficient.

11. The mechanical ventilator apparatus of claim 1, wherein the controller is configured to provide the test change by controlling the gas delivery apparatus to change an FIO₂ setting of the gas being delivered to the patient from a first FIO₂ level to a second FIO₂ level.

12. The mechanical ventilator apparatus of claim 11, wherein the controller is configured to determine the patient response time in response to determining that:
the first FIO₂ level is less than a max FIO₂ level; and
a SpO₂ level representative of the oxygen concentration of the patient's blood is less than a max SpO₂ level.

13. The mechanical ventilator apparatus of claim 11, wherein the controller is configured to determine the patient response time based at least in part on a period between a first time corresponding to the change in the FIO₂ setting from the first FIO₂ level to the second FIO₂ level and a second time corresponding to a specified response in the oxygen concentration of the patient's blood.

14. (canceled)

15. (canceled)

16. The mechanical ventilator apparatus of claim 13, wherein the period is a first period and the controller is further configured to:
control the gas delivery apparatus to change the FIO₂ setting from the second FIO₂ level to a third FIO₂ level;
determine a second period between a third time corresponding to the change in the FIO₂ setting from the second FIO₂ level to the third FIO₂ level and a fourth time corresponding to a specified response in the oxygen concentration of the patient's blood; and
determine the patient response time based at least in part on the first period and the second period.

17. The mechanical ventilator apparatus of claim 16, wherein the second FIO₂ level is greater than the first FIO₂ level, and the third FIO₂ level is less than the second FIO₂ level.

18. The mechanical ventilator apparatus of claim 13, wherein the specified response in the oxygen concentration of the patient's blood is based on a percentage of a total change in a measured SpO₂ level representative of the oxygen concentration of the patient's blood between the first time and a third time when the measured SpO₂ level reaches a steady state.

19. The mechanical ventilator apparatus of claim 18, wherein the SpO₂ level reaches a steady state when the SpO₂ level has not substantially changed over a given period of time.

20. The mechanical ventilator apparatus of claim 13, wherein the specified response in the oxygen concentration of the patient's blood is a peak response in a measured SpO₂ level representative of the oxygen concentration of the patient's blood.

21. The mechanical ventilator apparatus of claim 20, wherein the peak response in the measured SpO₂ level is determined using a regression-based model fit to data representative of the measured SpO₂ level.

22. The mechanical ventilator apparatus of claim 13, wherein the specified response in the oxygen concentration of the patient's blood is a given change in a measured SpO₂ level representative of the oxygen concentration of the patient's blood.

23. The mechanical ventilator apparatus of claim 22, wherein the given change in the measured SpO₂ level is determined using a regression-based model fit to data representative of the measured SpO₂ level.

24. The mechanical ventilator apparatus of claim 1, wherein the controller is further configured to determine if the oxygen concentration of the patient's blood has been below a threshold value for a predetermined period of time.

25. The mechanical ventilator apparatus of claim 24, wherein the controller is further configured to:
in response to determining that the oxygen concentration of the patient's blood has been below the threshold value for the predetermined period of time, control the gas delivery apparatus to change the FIO₂ setting to an FIO₂ level between 50% and 100%.

26. The mechanical ventilator apparatus of claim 1, wherein the oximetry sensor comprises a pulse oximetry sensor comprising an SpO₂ sensor.

27. The mechanical ventilator apparatus of claim 26, wherein the pulse oximetry sensor is configured for attachment to an ear or a finger of the patient.

28. The mechanical ventilator apparatus of claim 1, wherein the controller is configured to adjust the FIO₂ setting to cause a measured SpO₂ level representative of the oxygen concentration of the patient's blood to approach a target SpO₂ level.

29-83. (canceled)
